(19) United States
(12) Patent Application Publication
(10) Pub. No.: US 2002/0090352 A1
(43) Pub. Date: Jul. 11, 2002

(54) Compositions and methods for use in modulating immune system function

(76) Inventors: Frederick Geissmann, Paris (FR); Yves Lepelletier, Savigny-sur-Orge (FR); Michel Dy, Paris Cedex 15 (FR); Anne Durandy, Paris Cedex 15 (FR); Patrick Revy, Paris Cedex 15 (FR); Pierre Chambon, Blaesheim (FR)

Correspondence Address: STERNE, KESSLER, GOLDSTEIN & FOX PLLC, 1100 NEW YORK AVENUE, N.W., SUITE 600, WASHINGTON, DC, 20005-3934, US

(21) Appl. No.: 09/832,922

(22) Filed: Apr. 12, 2001

Related U.S. Application Data
(60) Provisional application No. 60/196,921, filed on Apr. 13, 2000.

(51) Int. Cl. A61K 38/19; A61K 31/07

(52) U.S. Cl. 424/85.1; 514/725

(57) ABSTRACT

Vitamin A (retinol) deficiency results in impaired response to infection and increased mortality. By the present invention, we show that retinol activates immature dendritic cells (DC) and enhances antigen presentation via a cross-talk with inflammatory cytokines, whereas it increases DC death in the absence of these cytokines. These effects, that are mediated through retinoic acids and distinct nuclear retinoid receptor pathways, can be dissociated from each other with selective synthetic retinoids. The present invention identifies a novel cellular target and function for retinoids, provides compositions and methods for modulating the immune system and for treating or preventing various physical disorders in animals, preferably via controlling activation and/or apoptosis in antigen-presenting cells using selective retinoids.

### CROSS-REFERENCE TO RELATED APPLICATIONS

The present application claims the benefit of U.S. Provisional Application No. 60/196,921, filed Apr. 13, 2000, the contents of which are entirely incorporated herein by reference.

### BACKGROUND OF THE INVENTION

1. Field of the Invention

The present invention relates to the fields of mammalian immunology, retinoid receptor biology and mammalian disease therapeutics. More particularly, the invention relates to compositions and methods useful in modulating the activity and function of the immune system of an animal. Specifically, the present invention provides compositions and methods for modulating the function of the immune system in an animal, particularly by activating antigen-presenting cells or by inhibiting/delaying apoptosis of antigen-presenting cells, the methods comprising administering to the animal an effective amount of a composition comprising at least one retinoid and at least one cytokine. In related aspects, the present invention provides compositions and methods for modulating the function of the immune system in an animal, particularly by inducing apoptosis of antigen-presenting cells, the methods comprising administering to the animal an effective amount of a composition comprising at least one synthetic retinoid. The methods and compositions of the present invention are useful in treating and/or preventing a variety of physical disorders in mammals including infectious (bacterial or viral) diseases, parasitic diseases, cancers (particularly carcinomas), immune system dysfunctions, and the like.

2. Related Art

### Retinoids

A number of studies have demonstrated that retinoids (vitamin A derivatives) are essential for normal growth, vision, tissue homeostasis, reproduction and overall survival (for reviews and references, See Sporn et al., The Retinoids, Vols. 1 and 2, Sporn et al., eds., Academic Press, Orlando, Fla. (1984)). For example, retinoids have been shown to be vital to the maintenance of skin homeostasis and barrier function in mammals (Fisher, G. J., and Voorhees, J. J., FASEB J. 10:1002-1013 (1996)). Retinoids are also apparently crucial during embryogenesis, since offspring of dams with vitamin A deficiency (VAD) exhibit a number of developmental defects (Wilson, J. G., et al., Am. J. Anat. 92:189-217 (1953); Morriss-Kay, G. M., and Sokolova, N., FASEB J. 10:961-968 (1996)). With the exceptions of those on vision (Wald, G., et al., Science 162:230-239 (1968)) and spermatogenesis in mammals (van Pelt, H. M. M., and De Rooij, D. G., Endocrinology 128:697-704 (1991)), most of the effects generated by VAD in animals and their fetuses can be prevented and/or reversed by retinoic acid (RA) administration (Wilson, J. G., et al., Am. J. Anat. 92:189-217 (1953); Thompson et al., Proc. Royal Soc. 159:510-535 (1964); Morriss-Kay, G. M., and Sokolova, N., FASEB J. 10:961-968 (1996)). The dramatic teratogenic effects of maternal RA administration on mammalian embryos (Shenefelt, R. E., Teratology 5, 103-108 (1972); Kessel, M., Development 115:487-501 (1992); Creech Kraft, J., In Retinoids in Normal Development and Teratogenesis, G. M. Morriss-Kay, ed., Oxford University Press, Oxford, UK, pp. 267-280 (1992)), and the marked effects of topical administration of retinoids on embryonic development of vertebrates and limb regeneration in amphibians (Mohanty-Hejmadi, et al., Nature 355:352-353 (1992); Tabin, C. J., Cell 66:199-217 (1991)), have contributed to the notion that RA may have critical roles in morphogenesis and organogenesis.

### Retinoid Receptors

Except for those involved in visual perception (Wald, G. et al., Science 162:230-239 (1968)), the molecular mechanisms underlying the highly diverse effects of retinoids have until recently remained obscure. The discovery of nuclear receptors for RA (Petkovich et al., Nature 330:444-450 (1987); Giguère et al., Nature 330:624-629 (1987)) has greatly advanced the understanding of how the retinoids may exert their pleiotropic effects (Leid et al., TIBS 17:427-433 (1992); Linney, E., Current Topics in Dev. Biol. 27:309-350 (1992)). Since this discovery it has become apparent that the genetic activities of the RA signal are mediated through two families of receptors—the RAR family and the RXR family—which belong to the superfamily of ligand-inducible transcriptional regulatory factors that include steroid/thyroid hormone and vitamin D3 receptors (for reviews see Leid et al., TIBS 17:427-433 (1992); Chambon, P., Semin. Cell Biol. 5:115-125 (1994); Chambon, P., FASEB J. 10:940-954 (1996); Giguère, V., Endocrinol. Rev. 15:61-79 (1994); Mangelsdorf, D. J., and Evans, R. M., Cell 83:841-850 (1995); Gronemeyer, H., and Laudet, V., Protein Profile 2:1173-1236 (1995)).

### RAR Receptors

Receptors belonging to the RAR family (RARα, β and γ and their isoforms) are activated by both all-trans- and 9-cis-RA (Leid et al., TIBS 17:427-433 (1992); Chambon, P., Semin. Cell Biol. 5:115-125 (1994); Dollé, P., et al., Mech. Dev. 45:91-104 (1994); Chambon, P., FASEB J. 10:940-954 (1996)). Within a given species, the DNA binding (C) and the ligand binding (E) domains of the three RAR types are highly similar, whereas the C-terminal domain F and the middle domain D exhibit no or little similarity. The amino acid sequences of the three RAR types are also notably different in their B regions, and their main isoforms (α1 and α2, β1 to β4, and γ1 and γ2) further differ in their N-terminal A regions (Leid et al., TIBS 17:427-433 (1992)). Amino acid sequence comparisons have revealed that the interspecies conservation of a given RAR type is greater than the similarity found between the three RAR types within a given species (Leid et al., TIBS 17:427-433 (1992)). This interspecies conservation is particularly striking in the N-terminal A regions of the various RARα, β and γ isoforms, whose A region amino acid sequences are quite divergent. Taken together with the distinct spatio-temporal expression patterns observed for the transcripts of each RAR and RXR type in the developing embryo and in various adult mouse tissues (Zelent, A., et al., Nature 339:714-717 (1989); Dollé, P., et al., Nature 342:702-705 (1989); Dollé et al., Development 110: 1133-1151 (1990); Ruberte et al., Development 108:213-222 (1990); Ruberte et al., Development 111:45-60 (1991); Mangelsdorf et al., Genes & Dev. 6:329-344 (1992)), this interspecies conservation has suggested that each RAR type (and isoform) may perform unique functions. This hypothesis is further supported by the finding that the various RAR isoforms contain two transcriptional activation functions (AFs) located in the N-terminal A/B region (AF-1) and in the C-terminal E region (AF-2), which can synergistically, and to some extent differentially, activate various RA-responsive promoters (Leid et al., TIBS 17:427-433 (1992); Nagpal, S., et al., Cell 70:1007-1019 (1992); Nagpal, S., et al., EMBO J. 12:2349-2360 (1993)).

### RXR Receptors

Unlike the RARs, members of the retinoid X receptor family (RXRα, β and γ) are activated exclusively by 9-cis-RA (Chambon, P., FASEB J. 10:940-954 (1996); Chambon, P., Semin. Cell Biol. 5:115-125 (1994); Dollé, P., et al., Mech. Dev. 45:91-104 (1994); Linney, E., Current Topics in Dev. Biol. 27:309-350 (1992); Leid et al., TIBS 17:427-433 (1992); Kastner et al., in Vitamin A in Health and Disease, R. Blomhoff, ed., Marcel Dekker, New York (1993)). However, the RXRs characterized to date are similar to the RARs in that the different RXR types also differ markedly in their N-terminal A/B regions (Leid et al., TIBS 17:427-433 (1992); Leid et al., Cell 68:377-395 (1992); Mangelsdorf et al., Genes and Dev. 6:329-344 (1992)), and contain the same transcriptional activation functions in their N-terminal A/B region and C-terminal E region (Leid et al., TIBS 17:427-433 (1992); Nagpal, S., et al., Cell 70:1007-1019 (1992); Nagpal, S., et al., EMBO J. 12:2349-2360 (1993)).

RXRα and RXRβ have a widespread (possibly ubiquitous) expression pattern during mouse development and in the adult animal, being found in all fetal and adult tissues thus far examined (Mangelsdorf, D. J., et al., Genes & Devel. 6:329-344 (1992); Dollé, P., et al., Mech. Devel. 45:91-104 (1994); Nagata,T., etal., Gene 142:183-189 (1994)). RXRγ transcripts, however, appear to have a more restricted distribution, being expressed in developing skeletal muscle in the embryo (where their expression persists throughout life), in the heart (after birth), in sensory epithelia of the visual and auditory systems, in specific structures of the central nervous system, and in tissues involved in thyroid hormone homeostasis, e.g., the thyroid gland and thyrotrope cells in the pituitary (Mangelsdorf, D. J., et al., Genes & Devel. 6:329-344 (1992); Dollé, P., et al., Mech. Devel. 45:91-104 (1994); Sugawara, A., et al., Endocrinology 136:1766-1774 (1995); Liu, Q., and Linney, E., Mol. Endocrinol. 7:651-658 (1993)).

It is currently unclear whether all the molecular properties of RXRs characterized in vitro are relevant for their physiological functions in vivo. In particular, it is unknown under what conditions these receptors act as 9-cis-RA-dependent transcriptional regulators (Chambon, P., Semin. Cell Biol. 5:115-125 (1994)). The knock-outs of RXRα and RXRβ in the mouse have provided some insight into the physiological functions of these receptors. For example, the ocular and cardiac malformations observed in RXRα^{-/-} fetuses (Kastner, P., et al., Cell 78:987-1003 (1994); Sucov, H. M., et al., Genes & Devel. 8:1007-1018 (1994)) are similar to those found in the fetal VAD syndrome, thus suggesting an important function of RXRα in the transduction of a retinoid signal during development. The involvement of RXRs in retinoid signaling is further supported by studies of compound RXRα/RAR mutants, which reveal defects that are either absent or less severe in the single mutants (Kastner, P., et al., Cell 78:987-1003 (1994); Kastner, P., et al., Cell 83:859-869 (1995)). Interestingly, however, knockout of RXRγ in the mouse induces no overt deleterious effects, and RXRγ^{-/-} homozygotes which are also RXRα^{-/-} or RXRβ^{-/-} exhibit no additional abnormalities beyond those seen in RXRα^{-/-}, RXRβ^{-/-} and fetal VAD syndrome fetuses (Krezel, W., et al., Proc. Natl. Acad. Sci. USA 93(17):9010-9014 (1996)), suggesting that RXRγ, despite its highly tissue-specific expression pattern in the developing embryo, is dispensable for embryonic development and postnatal life in the mouse. The observation that live-born RXRγ^{-/-}/RXRβ^{-/-}/RXRα^{+/-} mutants can grow to reach adult age (Krezel et al., Proc. Natl. Acad. Sci. USA 93(17):9010-9014 (1996)) indicates that a single RXRα allele is sufficient to carry out all of the vital developmental and postnatal functions of the RXR family of receptors, particularly all of the developmental functions which depend on RARs and may require RXR partnership (Dollé, P., et al., Mech. Dev. 45:91-104 (1994); Kastner, P., et al., Cell 83:859-869 (1995)). Furthermore, the finding that RXRα^{-/-}/RXRγ^{-/-} double mutant embryos are not more affected than are single RXRα^{-/-} mutants (Krezel et al., Proc. Natl. Acad. Sci. USA 93(1 7):9010-9014 (1996)) clearly shows that RXRβ alone can also perform some of these functions. Therefore, the fact that RXRα alone and, to a certain extent RXRβ alone, are sufficient for the completion of a number of developmental RXR functions, clearly indicates the existence of a large degree of functional redundancy amongst RXRs. In this respect, the RXR situation is different from that of RARs, since all of types of RAR double mutants displayed much broader sets of defects than single mutants (Rowe, A., et al., Develop. 111:771-778 (1991); Lohnes, D., et al., Develop. 120:2723-2748 (1994); Mendelsohn, C., Develop. 120:2749-2771 (1994)).

### Retinoid Binding to RAR and RXR Receptors

The crystal structures of the ligand-binding domains (LBDs) of the RARs and RXRs have recently been elucidated (Bourget, W., et al. , Nature 375:377-382 (1995); Renaud, J. P., et al., Nature 378:681-689 (1995); Wurtz, J. M., et al., Nature Struct. Biol. 3:87-94 (1996)). Among the various RAR types, substantial amino acid sequence identity is observed in these domains: comparison of the LBDs of RARα, RARα and RARγ indicates that only three amino acid residues are variable in the ligand-binding pocket of these receptors. These residues apparently account for the fact that the various RAR types exhibit some selectivity in binding certain synthetic retinoids (Chen, J. -Y., et al., EMBO J. 14(6):1187-1197 (1995); Renaud, J. P., et al., Nature 378:681-689 (1995)), and consideration of these divergent residues can be used to design RAR type-specific synthetic retinoids which may be agonistic or antagonistic (Chambon, P., FASEB J. 10:940-954 (1996)). This design approach may be extendable generally to other nuclear receptors, such as thyroid receptor a (Wagner, R. L., et al., Nature 378:690-697 (1995)), the ligand-binding pockets of which may chemically and structurally resemble those of the RARs (Chambon, P., FASEB J. 10:940-954 (1996)). Conversely, molecular modeling of the ligand-binding pocket of the RXRs demonstrates that there are no overt differences in amino acid composition between RXRα, RXRβ and RXRγ (Bourguet, W., et al., Nature 375:377-382 (1995); Wurtz, J. M., et al., Nature Struct. Biol. 3:87-94 (1996)), suggesting that design of type-specific synthetic ligands for the RXRs may be more difficult than for the RARs (Chambon, P., FASEB J. 10:940-954 (1996)).

### Retinoid Signaling Through RAR:RXR Heterodimers

Nuclear receptors (NRs) are members of a superfamily of ligand-inducible transcriptional regulatory factors that include receptors for steroid hormones, thyroid hormones, vitamin D3 and retinoids (Leid, M., et al., Trends Biochem. Sci. 1 7:427-433 (1992); Leid, M., et al., Cell 68:377-395 (1992); and Linney, E. Curr. Top. Dev. Biol., 27:309-350 (1992)). NRs exhibit a modular structure which reflects the existence of several autonomous functional domains. Based on amino acid sequence similarity between the chicken estrogen receptor, the human estrogen and glucocorticoid receptors, and the v-erb-A oncogene (Krust, A., et al., EMBO J. 5:891-897 (1986)), defined six regions—A, B, C, D, E and F—which display different degrees of evolutionary conservation amongst various members of the nuclear receptor superfamily. The highly conserved region C contains two zinc fingers and corresponds to the core of the DNA-binding domain (DBD), which is responsible for specific recognition of the cognate response elements. Region E is functionally complex, since in addition to the ligand-binding domain (LBD), it contains a ligand-dependent activation function (AF-2) and a dimerization interface. An autonomous transcriptional activation function (AF-1) is present in the non-conserved N-terminal A/B regions of the steroid receptors. Interestingly, both AF-1 and AF-2 of steroid receptors exhibit differential transcriptional activation properties which appear to be both cell type and promoter context specific (Gronemeyer, H. Ann. Rev. Genet. 25:89-123 (1991)).

As described above, the all-trans (t-RA) and 9-cis (9C-RA) retinoic acid signals are transduced by two families of nuclear receptors, RARα, β and γ (and their isoforms) are activated by both t-RA and 9C-RA, whereas RXRα, β and γ are exclusively activated by 9C-RA (Allenby, G. et al., Proc. Natl. Acad. Sci. USA 90:30-34 (1993)). The three RAR types differ in their B regions, and their main isoforms (α1 and α2, β1-4, and γ1 and γ2) have different N-terminal A regions (Leid, M. et al., Trends Biochem. Sci. 17:427-433 (1992)). Similarly, the RXR types differ in their A/B regions (Mangelsdorf, D. J. et al, Genes Dev. 6:329-344 (1992)).

The E-region of RARs and RXRs has also been shown to contain a dimerization interface (Yu, V.C. et al., Curr. Opin. Biotechnol. 3:597-602 (1992)). Most interestingly, it was demonstrated that RAR/RXR heterodimers bind much more efficiently in vitro than homodimers of either receptor to a number of RA response elements (RAREs) (Yu, V. C. et al., Cell 67:1251-1266 (1991); Berrodin, T. J. et al., Mol. Endocrinol 6:1468-1478 (1992); Bugge, T. H. et al., EMBO J. 11:1409-1418 (1992); Hall, R. K. et al., Mol. Cell. Biol. 12: 5527-5535 (1992); Hallenbeck, P. L. et al., Proc. Natl. Acad. Sci. USA 89:5572-5576 (1992); Husmann, M. et al., Biochem. Biophys. Res. Commun. 187:1558-1564 (1992); Kliewer, S. A. et al., Nature 355:446-449 (1992); Leid, M. et al., Cell 68:377-395 (1992); Marks, M. S. et al., EMBO J. 11:1419-1435 (1992); Zhang, X. K. et al., Nature 355:441-446 (1992)). RAR and RXR heterodimers are also preferentially formed in solution in vitro (Yu, V. C. et al., Cell 67:1251-1266 (1991); Leid, M. et al., Cell 68:377-395 (1992); Marks, M. S. et al., EMBO J. 11: 1419-1435 (1992)), although the addition of 9C-RA appears to enhance the formation of RXR homodimers in vitro (Lehman, J. M. et al., Science 258:1944-1946 (1992); Zhang, X. K. et al., Nature 358:587-591 (1992b)).

It has been shown that activation of RA-responsive promoters likely occurs through RAR:RXR heterodimers rather than through homodimers (Yu, V. C. et al., Cell 67:1251-1266 (1991); Leid et al., Cell 68:377-395 (1992b); Durand et al., Cell 71:73-85 (1992); Nagpal et al., Cell 70:1007-1019 (1992); Zhang, X. K., et al., Nature 355,441-446 (1992); Kliewer et al., Nature 355:446-449 (1992); Bugge et al., EMBO J. 11:1409-1418 (1992); Marks et al., EMBO J. 11:1419-1435 (1992); Yu, V. C. et al., Cur. Op. Biotech. 3:597-602 (1992); Leid et al., TIBS 17:427-433 (1992); Laudet and Stehelin, Curr. Biol. 2:293-295 (1992); Green, S., Nature 361:590-591 (1993)). The RXR portion of these heterodimers has been proposed to be silent in retinoid-induced signaling (Kurokawa, R., et al., Nature 371:528-531 (1994); Forman, B. M., et al., Cell 81:541-550 (1995); Mangelsdorf, D. J., and Evans, R. M., Cell 83:835-850 (1995)), although conflicting results have been reported on this issue (Apfel, C. M., et al., J. Biol. Chem. 270(51):30765-30772 (1995); see Chambon, P., FASEB J. 10:940-954 (1996) for review). Although the results of these studies strongly suggest that RAR/RXR heterodimers are indeed functional units that transduce the RA signal in vivo, it is unclear whether all of the suggested heterodimeric combinations occur in vivo (Chambon, P., Semin. Cell Biol. 5:115-125 (1994)). Thus, the basis for the highly pleiotropic effect of retinoids may reside, at least in part, in the control of different subsets of retinoid-responsive promoters by cell-specifically expressed heterodimeric combinations of RAR:RXR types (and isoforms), whose activity may be in turn regulated by cell-specific levels of all-trans- and 9-cis-RA (Leid et al., TIBS 17:427-433 (1992)).

The RXR receptors may also be involved in RA-independent signaling.

For example, the observation of aberrant lipid metabolism in the Sertoli cells of RXRβ^{-/-} mutant animals suggests that functional interactions may also occur between RXRβ and the peroxisomal proliferator-activated receptor signaling pathway (WO 94/26100; Kastner, P., et al., Genes & Devel. 10:80-92 (1996)).

### Antigen-presenting Cells

The introduction of non-self proteins into a eukaryotic cell or organism typically results in the appearance of peptide components of these non-self proteins in association with cell surface major histocompatability complex (MHC) molecules. These peptide/MHC composites are recognized by the immune system as “non-self,” resulting in the production of an immune response to the non-self protein. Certain cells involved in this immune response are capable of phagocytizing non-self proteins or organisms; these immune cells degrade or process the protein products, and the derived peptides are expressed at the surface of the cell in association with MHC molecules. Thereafter, a specific adaptive immune response is generated against the novel non-self components of the complexes. This activity of the immune cells is known as antigen processing and presentation, and cells that mediate this activity are typically referred to as antigen-presenting cells (see, e.g., WO 97/14426, WO 97/24447, WO 97/29182, and WO 97/29183, all of which are incorporated by reference herein in their entireties). A number of different immune cell types perform this function, including macrophages, dendritic cells, certain B cells, certain epithelial cells, and other associated cell types.

Dendritic cells (DC) are the major antigen-presenting cells in humans, and are critical for eliciting T cell-mediated immune responses (see Banchereau, J. and Steinman, R. M., Nature 392:245-252 (1998); WO 97/29182 and the references cited therein, all of which are incorporated by reference herein). Immature DC of the myeloid lineage, (e.g. Langerhans cells) are found in non-lymphoid tissues (such as the oral, bronchiolar, vaginal, rectal and skin epithelium). These cells have the unique capacity to (I) sample antigen at the boundary with the “external milieu”, (ii) migrate towards T-cell areas of the draining lymph node, (iii) mature to express costimulatory molecules such as CD96, and (iv) stimulate immune responses (Banchereau, J. and Steinman, R. M., Nature 392:245-252 (1998); Fernandez, N. C., et al., Nat. Med. 4:405-411 (1999); Kawano, T., et al., Science 278:1626-1629 (1997)). Recently, methods for generating pure populations of immature DC in vitro from peripheral blood monocytes have been described (Geissmann, F., et al., J. Exp. Med. 187:961-966 (1998); Geissmann, F., J. Immunol. 162:4567-4575 (1999)); these immature DC have the phenotype and functional characteristics of the Langerhans cell (referred herein to as LC). In addition, DC have been used as components in a variety of molecular-based (particularly gene therapy-based) approaches to developing new vaccination methods, including for the development of mammalian immune responses against tumor cell antigens for use in the treatment and prevention of certain cancers (see WO 97/14426; WO 97/24447; WO 97/29182; WO 97/29183; Paglia, P., et al., J. Exp. Med. 178:1893-1901 (1993); Paglia, P., et al., J. Exp. Med. 183:317-322 (1996); Steinman, R. M., Exp. Hematol. 24:859-862 (1996); Nestle, F. O., et al., Nature Med. 4:328-332 (1998), all of which are incorporated herein by reference in their entireties).

### Effects of Retinoids on the Immune System

Vitamin A (retinol, Rol) deficiency (VAD) results in impaired immune response to infection and increased mortality. Field studies have convincingly shown that retinol supplementation reduces mortality (by 20 to 30%) from infectious diseases among hundreds of million children in areas where VAD is endemic (Underwood, B. A. and Arthur, P., FASEB J. 10:1040-1048 (1996); Semba, R. D., Clin. Infect. Dis. 19:489-499 (1994); Rahmathullah, L., et al., N. Eng. J. Med. 323:929-935 (1990); Sommer, A., Lancet 339:864 (1992); Ross, A. C. and Stephensen, C. B., FASEB J. 10:979-985 (1996)). However, the cellular and molecular mechanisms that underlie the effect of Vitamin A on the immune system remains elusive, and high dose vitamin A supplementation in children—as currently recommended in developing countries—may have somewhat paradoxical effects (Semba, R. D. et al., Lancet. 345:1330-1332 (1995)).

Retinol is metabolized intracellularly along two distinct pathways forming (I) retinoic acids (RAs)—all-trans (tRA) and 9-cis (9cRA)—whose effects are transduced by nuclear retinoid receptors (RAR and RXR) (Chambon, P., FASEB. J. 10:940-954 (1996)); and (ii) retro-retinoids, which do not bind to known receptors (Buck, J., etal., Science 254:1654-1656 (1991)). Vitamin A has been shown to enhance B- and T-cell survival and proliferation in vitro (Buck, J., et al., Science 254:1654-1656 (1991); Garbe, A., et al., J. Exp. Med. 176:109-117 (1992); Buck, J. et al., J. Cell. Biol. 115:851-859 (1991)). These effects are triggered by retro-retinoids, and B- and T-cells neither respond to externally provided retinoic acid, nor synthesize it in appreciable amounts (Buck, J., et al., Science 254:1654-1656 (1991); Garbe, A., et al., J. Exp. Med. 176:109-117 (1992)). It seems reasonable to suggest that vitamin A may regulate other subsets of immune cells via a different pathway, as retinoic acid has been shown to either enhance or decrease immune responses (Dresser, D. W., Nature 217:527-529 (1968); Malkovsky, M., et al., Nature 302:338-340 (1983); Bedford, P. A. and Knight, S. C., Clin. Exp. Immunol. 75:481-486 (1989); Katz, D. R., et al., Br. J. Exp. Path. 68:343-350 (1987)), possibly through DC and LC which may be sensitive to its action in vivo (Bedford, P. A. and Knight, S. C., Clin. Exp. Immunol. 75:481-486 (1989); Katz, D. R., et al., Br. J. Exp. Path. 68:343-350 (1987); Walsh, L. J., et al., J. Invest Dermatol. 85:501-506 (1985); Meunier, L., et al., J. Invest. Dermatol. 103:775-779 (1994); Hachisuka, H. and Uno, H., Am. J. Dermatopathol. 9:316-323 (1987); Williams, N. A. and Hill, T. J., J. Invest. Dermatol. 97:933-937 (1991)). However, the question of whether or not retinoids may play a role in DC biology has not yet been definitively answered.

### Therapeutic Uses of Retinoids

As retinoic acid is known to regulate the capacities of several mammalian cell types to proliferate and/or differentiate (Gudas, L. J., et al., In The Retinoids, 2nd ed., Sporn, M. B., et al., eds., New York: Raven Press, pp. 443-520 (1994)), retinoids are used in a variety of chemopreventative and chemotherapeutic settings. The prevention of oral, skin and head and neck cancers in patients at risk for these tumors has been reported (Hong , W. K. et al., N. Engl. J. Med. 315:1501-1505 (1986); Hong, W. K. et al., N. Engl. J. Med. 323:795-801 (1990); Kraemer, K. H. et al., N. Engl. J. Med. 318:1633-1637 (1988); Bollag, W. et al., Ann. Oncol. 3:513-526 (1992); Chiesa, F. et al., Eur. J. Cancer B. Oral Oncol. 28:97-102 (1992); Costa, A. et al., Cancer Res. 54:Suppl. 7,2032-2037 (1994)). Retinoids have also been used to treat squamous cell carcinoma of the cervix and the skin (Verma, A. K., Cancer Res. 47:5097-5101 (1987); Lippman S. M. et al., J. Natl Cancer Inst. 84:235-241 (1992); Lippman S. M. et al., J. Natl Cancer Inst. 84:241-245 (1992)) and Kaposi's sarcoma (Bonhomme, L. et al., Ann. Oncol. 2:234-235 (1991)), and have found significant use in the therapy of acute promyelocytic leukemia (Huang, M. E. et al., Blood 72:567-572 (1988); Castaigne, S. et al., Blood 76:1704-1709 (1990); Chomienne, C. et al., Blood 76:1710-1717 (1990); Chomienne, C. et al., J. Clin. Invest. 88:2150-2154 (1991); Chen Z. et al., Leukemia 5:288-292 (1991); Lo Coco, F. et al., Blood 77:1657-1659 (1991); Warrell, R. P., et al., N. Engl. J. Med. 324:1385-1393 (1991); Chomienne, C., et al., FASEB J. 10: 1025-1030 (1996)).

Despite extensive knowledge of RA action at the molecular level and the use of retinoids in treating a variety of physical disorders, however, the cellular mechanisms that underlie the effects of vitamin A on the immune system remain elusive. The present invention identifies mechanisms by which retinoids modulate the activity of the immune system, and provides therapeutic and preventative methods and compositions acting through these mechanisms to regulate immune system function and to treat and/or prevent certain mammalian physical disorders.

### BRIEF SUMMARY OF THE INVENTION

By the invention, methods and compositions are provided for modulating the activity of the immune system in an animal, preferably to prevent or treat a physical disorder in the animal via such modulation. In particular, the methods and compositions of the invention may be used to differentially modulate the response to one or more antigens by certain components of the immune system, particularly antigen-presenting cells such as dendritic cells, Langerhans cells, macrophages, and the like. In certain such embodiments, the invention provides methods of modulating the immune system of an animal by affecting the physiology of an antigen-presenting cell in the animal, for example by a method comprising contacting the antigen-presenting cell with an effective amount of at least one retinoid and an effective amount of at least one cytokine, under conditions whereby the physiology of the antigen-presenting cell is affected.

The methods and compositions of the invention are based in part upon the present an unexpected discovery that retinoids and cytokines may act cooperatively and, in some cases, synergistically, to positively modulate the immune system in animals, particularly by affecting the phyisology of antigen- presenting cells in ways such as activating the cells or inhibiting or delaying apoptosis of the cells. In addition, the methods and compositions of the invention are based in part upon the discovery that specific and selective retinoids, particularly selective synthetic retinoids, may negatively modulate the immune system in animals, particularly by affecting the physiology of antigen-presenting cells such as by inducing apoptosis of such cells. According to the invention, preferred retinoids for use in the present methods and compositions include one or more RAR agonists (particularly one or more RARα agonists such as 4-[[(2,3-Dihydro-1,1,3,3-tetramethyl-2-oxo-1H-inden-5-yl)carbonyl]amino]benzoic acid (referred to herein as “Compound I” and the structure and synthesis of which is shown below and in WO 98/47861, which is incorporated herein by reference), one or more RXR agonists (particularly one or more pan-RXR agonists such as SR11237 (4[2-(5,6,7,8-tetrahydro-5,5,8,8-tetramethyl-2-naphthalenyl)-1,3-dioxolan-2-yl]benzoic acid, the structure and synthesis of which are disclosed in U.S. Pat. No. 5,552,271 which is incorporated herein by reference) and/or 4-[1-[5,6-Dihydro-3,5,5-trimethyl-8-(1 -methylethyl)-2-naphthalenyl]-ethenyl]benzoic acid (referred to herein as “Compound V” and the structure and synthesis of which are provided in commonly owned, co-pending U.S. application Ser. Nos. 60/127,976, filed Apr. 6, 1999, and 60/130,649, filed Apr. 23, 1999, which are incorporated herein by reference), one or more RAR antagonists (particularly one or more pan-RAR antagonists such as 4-[(1E)-2-[5,6-dihydro-5,5-dimethyl-8-phenylethynyl)-2-naphthalenyl]-ethenyl]benzoic acid (referred to herein as “Compound VIII” and the structure and synthesis of which are provided in WO 98/46228, which is incorporated herein by reference)) or one or more RARα antagonists such as 4-[[[5,6-Dihydro-5,5-dimethyl-8-(3-quinolinyl)-2-naphthalenyl]carbonyl]-amino]benzoic acid (referred to herein as “Compound II” and the structure and synthesis of which is disclosed in U.S. Pat. Nos. 5,559,248 and 5,849,923, which are incorporated herein by reference)), one or more RXR antagonists. As will be recognized by those of ordinary skill in the pharmaceutical arts, of course, the methods and compositions of the invention also encompass other forms of the compounds described herein, such as (where available) pharmaceutically acceptable salts, esters, prodrugs, variants, and derivatives of the retinoids specifically described herein. In certain embodiments, the methods and compositions of the invention may advantageously use or comprise one or more cytokines. Preferred cytokines for use in the methods and compositions of the invention include any compound that induces a physiological response in a cell, such as growth, differentiation, senescence, apoptosis, cytotoxicity or antibody secretion, including but not limited to growth factors, interleukins, colony-stimulating factors, interferons, and the like. Particularly preferred cytokines for use in accordance with the present invention are IL-1β, TNFα, and active fragments, variants and derivatives thereof. The nucleotide sequences for the TNFα and IL1β, genes are available under GenBank Accession Nos. E02870 and X04500, respectively, which are incorporated herein by reference in their entireties.

Thus, in one aspect the invention provides methods of modulating the immune system of an animal, preferably a mammal including but not limited to a human, comprising affecting the physiology of an antigen-presenting cell in the animal. In certain preferred such embodiments, the methods of the invention comprise activating an antigen-presenting cell preferably by contacting the antigen-presenting cell with one or more retinoids and one or more cytokines, or with one or more of the compositions of the invention described hereinbelow. As used herein, a compound or composition is said to “affect the physiology of” an antigen-presenting cell if it alters or otherwise modulates (positively or negatively) one or more physiological processes in the cell relative to the level of those particular physiological processes in an antigen-presenting cell that has not been contacted by the compound or composition. For example, activation of an antigen-presenting cell may be indicated by an increase in antigen presentation by the cell (e.g., an increase in amount, efficiency, or speed of antigen processing and cell surface display of the antigen by the antigen-presenting cell; an increase in DNA synthesis (measured, e.g., by determining the uptake and incorporation into cellular DNA of one or more labeled nucleotides) in an allogenous or autologous T cell in response to an antigen presented by an activated antigen-presenting cell), and/or an induction of differentiation or maturation of an undifferentiated or immature antigen-presenting cell (measured, e.g., by an increase in the expression of one or more cellular markers of differentiation or maturation, for example one or more cell surface CD antigens). Retinoids useful according to this aspect of the invention include any retinoid that activates an antigen-presenting cell, particularly pan-RXR agonists (such as SR11237 or Compound V) and pan-RAR antagonists (such as Compound V). In certain such methods of the invention, the antigen-presenting cells may optionally also be contacted with an effective amount of Compound II. Preferred cytokines for use in accordance with this aspect of the invention include any cytokines that act cooperatively or synergistically with one or more retinoids to activate antigen-presenting cells, particularly TNFα and IL-1β, and most particularly TNFα. Particularly preferred compositions for use in accordance with this aspect of the invention include, for example, compositions comprising: (a) an effective amount of each of Compound V and TNFα; (b) an effective amount of each of SR1 1237 and TNFα; or (c) an effective amount of each of SR1 1237, Compound II and TNFα; or pharmaceutically acceptable salts thereof. According to the invention, the antigen-presenting cell may be contacted with the one or more retinoids and one or more cytokines simultaneously or sequentially, in any order, in amounts effective to activate the antigen-presenting cell. Related methods for activating an antigen-presenting cell may comprise contacting the cell with one or more of the pharmaceutical compositions of the invention described hereinbelow. The antigen-presenting cell may be contacted with the one or more retinoids and one or more cytokines, or with the pharmaceutical compositions, in vivo, in vitro, or ex vivo.

In a related aspect, the invention provides methods of screening candidate compounds or compositions to select a compound or composition capable of activating an antigen-presenting cell. Preferred such methods may comprise, for example:

(a) obtaining a first and a second mammalian antigen-presenting cells;

(b) contacting the first cell, but not the second cell, with one or more compositions to be assayed for the ability to activate an antigen-presenting cell;

(c) determining the level of expression of a cellular phenotype associated with activation in the first and second cells;

(d) comparing the level of the cellular phenotype in the first and second cells; and

(e) selecting a composition wherein the level of the cellular phenotype in said first antigen-presenting cell is different from the level of the cellular phenotype in the second antigen-presenting cell, wherein a difference in the phenotype between the first and second cells is indicative of induction of activation in the first cell.

In another aspect, the invention provides methods of modulating the immune system of an animal, preferably a mammal including but not limited to a human, by affecting the physiology of an antigen-presenting cell in the animal, for example by methods comprising inhibiting, delaying or preventing apoptosis in an antigen-presenting cell, particularly retinoid-induced apoptosis of an antigen-presenting cell. Preferred such methods of the invention may comprise, for example, contacting the antigen-presenting cell with one or more retinoids and one or more cytokines, or with one or more of the compositions of the invention described hereinbelow, in amounts effective to inhibit, delay or prevent the apoptosis (particularly retinoid-induced apoptosis) of an antigen-presenting cell. As used herein, a compound or composition is said to “inhibit, delay or prevent” apoptosis in an antigen-presenting cell if it causes a decrease in, or delays the onset of, or prevents, one or more physiological processes or characteristics in the cell that are associated with apoptosis, relative to the level of those particular physiological processes or characteristics in an antigen-presenting cell that has not been contacted by the compound or composition. For example, apoptosis of an antigen-presenting cell may be indicated by any phenotype, marker or activity that may be observed to change (i.e., increase or decrease) in expression, level or amount in an antigen-presenting cell that is undergoing apoptosis compared to an antigen-presenting cell that is not undergoing apoptosis. Such phenotypes, markers or activities may include, but are not limited to, DNA fragmentation (determination of which may be accomplished by methods such as gel electrophoresis (appearance of smaller molecular weight bands)), changes in microscopic phenotype (e.g., changes in plasma membrane morphology such as formation of surface protuberances (“blebbing”) or in nuclear morphology such as pycnosis or fragmentation), a decrease in the expression of the putative apoptosis suppressive protein BCL-2 (decreased in apoptotic cells), an increase in cell surface expression of annexin V (increased in apoptotic cells; see Example 1 herein), the appearance in a cell population of smaller cells with different light scatter and/or DNA content profiles as assessed, for example, by flow cytometry or particle analysis, and other methods that are well-known in the art. Retinoids useful according to this aspect of the invention include any retinoid that inhibits, delays or prevents apoptosis in an antigen-presenting cell, particularly RAR agonists (more particularly RARα agonists such as Compound I), RAR antagonists (more particularly RARα antagonists such as Compound II, or pan-RAR antagonists such as Compound V or Compound VIII), and RXR agonists (particularly pan-RXR agonists such as SR11237 or Compound V). Preferred cytokines for use in accordance with this aspect of the invention include any cytokines that act cooperatively or synergistically with one or more retinoids to inhibit, delay or prevent apoptosis in antigen-presenting cells, particularly TNFα and IL-1β, and most particularly TNFα. Particularly preferred compositions for use in accordance with this aspect of the invention include, for example, compositions comprising: (a) an effective amount of Compound II; (b) an effective amount of each of Compound II and SR11237; (c) an effective amount of Compound V; (d) an effective amount of each of Compound V and SR11237; (e) an effective amount of Compound VIII; (f) an effective amount of each of Compound VIII and SR11237; or (g) an effective amount of each of TNFα and SR11237; or pharmaceutically acceptable salts thereof. According to the invention, the antigen-presenting cell may be contacted with the one or more retinoids and one or more cytokines simultaneously or sequentially, in any order, in vivo, in vitro, or ex vivo, in amounts effective to inhibit, delay or prevent apoptosis in the antigen-presenting cell. Related methods for inhibiting, delaying or preventing apoptosis in an antigen-presenting cell may comprise contacting the cell with one or more of the pharmaceutical compositions of the invention described hereinbelow.

In related aspects, the invention provides methods for screening candidate compounds in order to identify compounds capable of inhibiting, delaying or preventing apoptosis in an antigen-presenting cell. One such method may, for example, comprise:

(a) contacting a first and a second antigen-presenting cell with one or more compounds that induce apoptosis in the antigen-presenting cell (preferably one or more apoptosis-inducing retinoids such as one or more RARα agonists (e.g., tRA, 9cRA, or Compound I (see below), optionally in conjunction with one or more RXR agonists (e.g., SR11237, a pan-RXR agonist)), under conditions favoring the development of one or more cellular characteristics of apoptosis in the cell;

(b) contacting the first, but not the second, cell with one or more test compounds to be screened for the ability to inhibit, delay or prevent apoptosis in the first cell; and

(c) measuring the levels of one or more cellular characteristics of the apoptotic phenotype in the first and second cells, wherein a reduction in the level of such characteristics in the first cell relative to those in the second cell indicates that the test compound is able to inhibit, delay or prevent apoptosis in an antigen-presenting cell.

In another aspect, the invention provides additional methods of modulating the immune system of an animal, preferably a mammal including but not limited to a human, by affecting the physiology of an antigen-presenting cell. Methods according to this aspect of the invention may comprise, for example, inducing apoptosis of an antigen-presenting cell. Preferred such methods of the invention may comprise, for example, contacting the antigen-presenting cell with one or more apoptosis-inducing retinoids, or with one or more of the compositions of the invention described hereinbelow, in amounts effective to induce apoptosis in an antigen-presenting cell. As used herein, a compound or composition is said to “induce” apoptosis in an antigen-presenting cell if it causes an increase in, or accelerates the onset of, one or more physiological processes or characteristics in the cell that are associated with apoptosis, relative to the level of those particular physiological processes or characteristics in an antigen- presenting cell that has not been contacted by the compound or composition. For example, apoptosis of an antigen-presenting cell may be indicated by any phenotype, marker or activity that may be observed to change (i.e., increase or decrease) in expression, level or amount in an antigen-presenting cell that is undergoing apoptosis compared to an antigen-presenting cell that is not undergoing apoptosis. Such phenotypes, markers or activities may include, but are not limited to, those described above and in the Examples below. Retinoids useful according to this aspect of the invention include any retinoid that induces apoptosis in an antigen-presenting cell, particularly RAR agonists (more particularly RARα agonists such as Compound I, and RARβ agonists such as Compound m or Compound VII), and RXR agonists (more particularly pan-RXR agonists such as SR11237 or Compound V). Particularly preferred compositions for use in accordance with this aspect of the invention include, for example, compositions comprising: (a) an effective amount of Compound I; (b) an effective amount of each of Compound I and SR11237; (c) an effective amount of Compound III; (d) an effective amount of each of Compound III and SR11237; (e) an effective amount of Compound IV; (f) an effective amount of each of Compound IV and SRI 1237; (g) an effective amount of Compound VII; or (h) an effective amount of each of Compound VII and SR11237; or pharmaceutically acceptable salts thereof. According to the invention, the antigen-presenting cell may be contacted with the one or more retinoids (or compositions) in vivo, in vitro, or ex vivo, in amounts effective to induce apoptosis in the antigen-presenting cell. Related methods for inducing apoptosis in an antigen-presenting cell may comprise contacting the cell with one or more of the pharmaceutical compositions of the invention described hereinbelow.

In a related aspect, the invention provides methods for screening candidate compounds in order to identify compounds capable of inducing the apoptosis of an antigen-presenting cell. One such method, for example, may comprise:

(a) obtaining a first and a second antigen-presenting cell;

(b) contacting the first, but not the second, cell with one or more test compounds to be screened for the ability to induce apoptosis in the first cell; and

(c) measuring the levels of one or more cellular characteristics of the apoptotic phenotype in the first and second cells, wherein an increase in the level of such characteristics in the first cell relative to those in the second cell indicates that the test compound is able to induce apoptosis in an antigen-presenting cell.

In related aspects, the invention provides compositions, particularly pharmaceutical compositions, that may be used to treat or prevent a physical disorder in an animal (particularly in a mammal, including a human) suffering from, or predisposed or susceptible to, the physical disorder. While not wishing to be bound by any mechanistic explanation for their therapeutic or prophylactic effectiveness, it is presumed that these compositions of the invention are effective in modulating the immune system of an animal, preferably a mammal such as a human, by affecting the physiology of (e.g., by activating, or inhibiting/preventing/delaying or inducing apoptosis in) antigen-presenting cells in the animal. Such compositions according to this aspect of the invention preferably comprise one or more retinoids (particularly those described herein as having modulating effects upon the immune system via affecting activation and/or apoptosis in antigen-presenting cells) and optionally one or more cytokines (particularly for those compositions used for activation of, or inhibiting/delaying/preventing apoptosis in, antigen-presenting cells), and may further optionally comprise a pharmaceutically acceptable carrier, diluent or excipient therefor.

Certain compositions of the invention may further optionally comprise one or more additional components, such as one or more pharmaceutically acceptable carriers, diluents or excipients as described herein. Other preferred compositions of the invention, particularly those that may be useful as vaccine conjugates for use in vaccinating an animal to treat or prevent a particular disorder or disease, may optionally comprise one or more antigens. Antigens suitable for inclusion in such compositions of the invention may be any antigen to which an immune response is to be raised in the animal, including, for example, one or more bacterial antigens, one or more fungal antigens, one or more viral antigens, one or more animal antigens (including one or more parasite antigens), one or more tumor cell antigens (which may or may not be one or more tumor cell-specific antigens), one or more plant antigens, or any combination(s) thereof.

In related aspects, the invention provides methods of treating or preventing a physical disorder in an animal (particularly in a mammal, including a human) suffering from, or predisposed or susceptible to, the physical disorder. Methods according to this aspect of the invention may comprise, for example, administering to the animal an effective amount of one or more retinoids and one or more cytokines. According to the invention, the one or more retinoids and one or more cytokines may be administered to the animal simultaneously or sequentially, in any order, in dosages designed to provide the desired therapeutic or preventative benefit while minimizing undesired or untoward side-effects. Related methods may comprise administering to the animal one or more of the pharmaceutical compositions of the invention.

The methods and compositions of the invention are useful in treating or preventing a variety of physical disorders in a variety of animals, particularly in mammals including humans, that are suffering from, or predisposed or susceptible to, the physical disorders. Physical disorders treatable or preventable using the compositions and methods of the invention include any physical disorder that is treatable or preventable via modulation of the immune system of the animal, particularly via affecting the physiology of antigen-presenting cells in the animal, such as by inducing activation, inhibiting, delaying or preventing apoptosis, or activating apoptosis, of antigen-presenting cells. Such physical disorders may include, but are not limited to, infectious diseases (particularly bacterial, viral and/or fungal diseases), parasitic diseases, cancers (such as carcinomas, melanomas, sarcomas and the like), immune system disorders (such as rheumatoid arthritis, multiple sclerosis, systemic lupus erythematosis, Crohn's Disease), and the like. According to a preferred such aspect, the methods and compositions of the invention may be used to induce or enhance an immune response to one or more antigens associated with a physical disorder in an animal. In one such aspect, for example, the compositions of the invention may be administered to an animal in need thereof as a vaccine formulation, for example in conjunction with (i.e., in admixture with, or as a separate administration, e.g., as an adjuvant) one or more antigen-containing formulations or compositions, such as compositions comprising one or more bacterial antigens, one or more viral antigens, one or more fungal antigens, one or more parasite antigens, and the like, whereby the administration of such compositions induces or enhances an immune response of the animal to the one or more antigens, thus treating or preventing a physical disorder with which the one or more antigens is associated.

According to the invention, the antigen-presenting cells used or contacted in the present methods may be any antigen-presenting cell, including but not limited to dendritic cells, Langerhans cells, macrophages, and the like. Antigen-presenting cells may be contacted with the compositions or compounds in the above-described methods in vitro, ex vivo, or in vivo.

Other preferred embodiments of the present invention will be apparent to one of ordinary skill in light of the following drawings and description of the invention, and of the claims.

### BRIEF DESCRIPTION OF THE DRAWINGS

FIG. 1. Retinoids induce death of immature LC, but increase MHC class II and costimulatory CD86 molecule expression as well as alloreactive proliferative response in the presence of TNFα.

FIG. 1A: Survival of immature LC in presence of retinol. Day 6 immature LC were cultured in presence of increasing amounts of retinol (Rol) and viable cells were counted each day in malassez with trypan blue exclusion (mean of three experiments on different donors ±SD; *p<0.05).

FIG. 1B: Apoptosis of immature LC is increased in presence of retinol and inhibited by an RAR antagonist. Immature LC were cultured with retinol and/or the pan-RAR(αβγ) antagonist Compound VIII or vehicle for 40 h. Cells were washed and incubated with anti-annexin V antibody and propidium iodide (PI) 2 μg/ml. 10⁴ total events (without gating) were then analyzed, with a FACScalibur (Becton Dickinson) using CellQuest software (Becton Dickinson). Data are representative of 3 experiments on different donors.

FIG. 1C: Retinol- and TNFα-treated LC increase alloreactive proliferative response. Immature LC were cultured with TNFα, and/or retinol or vehicle for 40 h. Cells were washed 4 times in RPMI with 10% human AB serum and added in triplicate at stimulator/effector ratio of 1% and 4% to 10⁵ purified T-cells/well from the same donor or from a second donor, in 96-well tissue culture plates. SD are indicated; *:p<0.05.

FIG. 1D: Retinol and TNFα, synergize to increase MHC class II and costimulatory CD86 expression. Immature LC were cultured with retinol or vehicle and/or TNFα for 40 h. Cells were washed and incubated with HLA-DR-FITC and CD86-PE or isotype controls for 15 min at 4° C. 10⁴ events were then analyzed with a FACScalibur (Becton Dickinson) using CellQuest software (Becton Dickinson). % are given for DRhi CD86hi cells gated as indicated. Data are representative of three experiments on different donors.

FIG. 1E: Retinol and tRA synergize with TNFα to induce a dose-dependent increase in the percentage of DR^{hi}-CD86^{hi} LC which parallels inhibition of apoptosis. Cells were cultured as indicated above. Expression of HLA DR and CD86 (see above) and apoptosis (%<2N particle; see Table 3) were determined at the same time point on separate aliquots of the same culture.

FIG. 2. Induction of Apoptosis by Natural and Synthetic Retinoids.

FIG. 2A: Survival of immature LC in presence of tRA and synthetic RARα ligands (a), (b): Day 6 immature LC were cultured in the absence (●) or the presence of increasing amounts (10Δ, 100▴, 1000 nM ◯) of tRA (panel a) or RARα agonist Compound I (panel b) and viable cells were counted each day with trypan blue exclusion. (c) In the presence of 1000 nM tRA (◯), the addition of the RARα antagonist Compound II (1000 nM, ▪) inhibited mortality by 50%, while the addition of the caspase-inhibitor Z-Vad-fmk (50 nM, □) inhibited tRA mortality by >80%. (●): Survival of untreated cells. Mean±SD of three experiments on different donors. * p<0.05.

FIG. 2B: Dose dependent-tRA induced apoptosis. Cells were processed as indicated above (see FIG. 1B).

FIG. 3.

FIG. 3A: Day 6 immature LC were cultured for 40 h with TNFα and/or retinoids or vehicle as indicated. Cells were washed and stained with HLA-DR-FITC and CD86-PE or isotype controls for 15 min at 4° C. 10⁴ events were then analyzed with a FACScalibur (Becton Dickinson) using CellQuest software (Becton Dickinson). Data are given for DRhi/CD86 hi cells gated as indicated, and are representative of 10 experiments on different donors.

FIG. 3B: Retinol (●), tRA (▪), 9cRA (□), the RARα agonist Compound I (◯), the RARβ agonist Compound III (Δ), and the RXR agonist SR11237 (▴), upregulate MHC class II and costimulatory CD86 molecules on immature LC in the presence of TNFα. The percentages of DRhi/CD86 hi cells were plotted against increasing concentrations (nM) of each retinoids. Results correspond to a representative experiment.

FIGS. 3C and 3D: Cells were cultured in the presence of TNFα (10 ng/ml) with 100 nM tRA, Compound I or 9cRA, or with 1000 nM SR11237, with or without the pan-RAR antagonist Compound VIII (1000 nM) or the RARα ligand Compound II (1000 nM). Percentages of DRhi/CD86 hi cells are represented and results are expressed as the mean±SD of at last three experiments on different donors.

FIG. 3E: Cells were cultured in the presence of TNFα (10 ng/ml) with SR11237 (1000 nM) and increasing doses (nM) of Compound II (●) or Compound VIII (◯), or, as controls, with Compound I (100 nM) and increasing doses (nM) of Compound VIII (□), or with vehicle (ethanol), and increasing doses (nM) of Compound II (▪). The percentages of DRhi/CD86 hi cells are presented. Results are from one representative experiment.

FIG. 4.

FIG. 4A: Retinoids increase antigen presentation by immature LC. Day 6 immature LC were pulsed for 8 h with tetanus toxoid (TT) or medium alone, and then cultured with TNFα, and/or retinoids for 40 h. Cells were then washed 4 times in medium containing 10% human AB serum, and added at stimulator/effector ratio of 4% and 16% to 10⁵ purified T-cells/well from the same donor, in triplicate in 96-well tissue culture plates. T-cell proliferation was measured as indicated in materials and methods. Background thymidine incorporation, in the absence of pulse with tetanus toxin, is indicated (no TT) and was always less than 10% of the antigen specific response). SD were <15%. *:p<0.01.

FIG. 4B: Retinoid treated LC increase alloreactive proliferative response.

Day 6 immature LC (LC like) were cultured with TNFα and/or retinoids or vehicle (ethanol) for 40 h. Cells were then washed 4 times in medium containing 10% human AB serum and added in triplicate at stimulator/effector ratio of 1% and 4% to 10⁵ purified T-cells/well from the same donor or from a second donor, in 96-well tissue culture plates. Background thymidine incorporation was always less than 10% of alloreactive response. SD were <15%. *:p<0.01.

FIG. 4C: Production of IL-12 upon CD40 triggering. Day 6 immature LC were incubated as indicated for 40 hours with tRA (100 nM), the RXR agonist SR11237 (1000 nM) and/or TNFα (10 ng/ml), and either CD40-L transfected fibroblasts or CD32-transfected fibroblasts as control. Supernatants were then collected and were analyzed for bioactive p70 IL-12 production using ELISA. Results are mean and SD of 5 experiments on different donors.

FIG. 5. tRA, The RARα agonist Compound I, the RXR agonist SR11237, but not vehicle (EtOH) or the RARα antagonist Compound II synergize with TNFα to induce nuclear translocation of NF-kB. Day 6 immature LC (LC like) were washed and incubated for 6 h in fresh complete medium alone, or with vehicle (ethanol) or retinoids at a 1 μM concentration. Cells were then washed and incubated for 30 min with TNFα (50 ng/ml) or medium alone as control.

Then, nuclear extracts were incubated for 30 minutes at 37° C. with the double-stranded labeled probes for NF-kB, loaded onto a non denaturing 5% polyacrylamide gel and subjected to electrophoresis at 14 V/cm in a low-ionic-strength buffer (0.5×TBE). Gels were dried and examined with a Phosphorimager (Molecular Dynamics, Sunnyvale, Calif.).

FIG. 6. Schematic representation of retinoid receptor pathways that transduce apoptosis or activation of immature dendritic cells (Langerhans cells).

### DETAILED DESCRIPTION OF THE INVENTION

### Definitions

In the description that follows, a number of terms conventionally used in the fields of pharmacology, immunology, retinoid biology and molecular biology are utilized extensively. In order to provide a clear and consistent understanding of the specification and claims, and the scope to be given such terms, the following definitions are provided.

The term “cytokine” refers to a compound that acts as a biological response modifier to coordinate antibody and T cell immune system interactions and amplify immune reactivity (see, Abbas, A.K., et al., CELLULAR AND MOLECULAR IMMUNOLOGY, 2nd ed., 1994). As such, cytokines induce one or more physiological responses in a target cell, such as growth, differentiation, senescence, apoptosis, cytotoxicity or antibody secretion. Included in this definition of “cytokine” are growth factors, interleukins, colony-stimulating factors, interferons and lymphokines, which may be natural, synthetic, or recombinant. Also included are analogues or homologues of such molecules, which may also be natural, synthetic or recombinant (see, e.g., Novaković et al., Cytokine 9:597-604 (1997), which is incorporated by reference herein, for a discussion of preparation and activity of TNFα analogues).

As used herein, “antigen-presenting cell” refers to any cell, regardless of the tissue derivation or source of the cell, that is involved in certain aspects of the immune response of an organism, particularly those cells that are capable of phagocytizing non-self proteins or organisms, degrading or processing the protein products, and expressing the derived peptides at the surface of the cell in association with MHC molecules. Thereafter, a specific adaptive immune response is generated against the novel non-self components of the complexes. Antigen-presenting cells are any cells capable of carrying out the process of antigen processing and presentation, including but not limited to macrophages (including tissue-fixed macrophages, such as Kupffer cells, histiocytes, etc.), dendritic cells (including immature dendritic cells such as Langerhans cells), monocytes (and monocyte-derived antigen-presenting cells such as monocyte-derived macrophages), certain B cells, certain antigen-presenting epithelial cells, and the like.

The term “retinoid” includes vitamin A derivatives, analogues, homologues and the like which are capable of binding to one or more retinoid receptors on the surface of a cell. Retinoids include not only naturally derived compounds and compositions (e.g., 9-cis-retinoic acid and all-trans-retinol), but also synthetic compounds and compositions that have the retinoid receptor binding and activity characteristics described herein. The term “retinoid” thus refers not only to those compounds that bind to a retinoid receptor and directly or indirectly induce, increase, up-regulate or otherwise positively modulate some activity in the cell (referred to as an “agonist” of the receptor), but also to those compounds that bind to a retinoid receptor and directly or indirectly inhibit, reduce, down-regulate or otherwise negatively modulate some activity in the cell (referred to as an “antagonist” of the receptor). Of course, as one of ordinary skill in the applicable arts will readily understand, the term “retinoid” as used herein refers not only to the specific compounds described herein, but also to pharmaceutically acceptable salts, variants, and derivatives thereof.

Other terms and phrases used herein will be understood by those of ordinary skill as having the meanings conventionally associated with those terms and phrases in the relevant arts, unless otherwise indicated herein in ipsis verbis or by the context in which those terms and phrases may be used.

### Overview

The present invention provides methods and compositions for modulating the activity of the immune system in an animal, preferably to prevent or treat a physical disorder in the animal via such modulation. In particular, the methods and compositions of the invention may be used to affect the physiology of, or differentially modulate the response to one or more antigens by, certain components of the immune system, particularly a variety of antigen-presenting cells such as dendritic cells, Langerhans cells, macrophages, and the like. Preferred such methods of the invention include, for example: (a) activating antigen-presenting cells; (b) inhibiting, delaying or preventing apoptosis in antigen-presenting cells; or (c) inducing apoptosis in antigen-presenting cells.

Thus in one aspect, the invention provides methods of modulating the immune system of an animal (preferably a mammal such as a human) by affecting the physiology of an antigen-presenting cell, preferably by activating an antigen-presenting cell, comprising contacting the antigen-presenting cell with one or more retinoids and one or more cytokines, or with one or more of the compositions of the invention, under conditions whereby the physiology of the antigen-presenting cell is affected. Such conditions may comprise, for example, contacting the antigen-presenting cell with the one or more retinoids and one or more cytokines, or with the one or more compositions of the invention, in amounts effective to affect the physiology of the antigen-presenting cell. In one such aspect, the methods comprise activating the antigen-presenting cell by contacting the cell with one or more retinoids and one or more cytokines, or with one or more compositions of the invention, in amounts effective to activate the antigen-presenting cell. Preferred methods and compositions for use in accordance with this aspect of the invention include contacting the cell with at least one pan-RXR agonist (e.g., SR11237 or Compound V) or at least one pan-RAR antagonist (e.g., Compound V), or pharmaceutically acceptable salts thereof. Additional such embodiments may further comprise contacting the antigen-presenting cell with an effective amount of Compound II or a pharmaceutically acceptable salt thereof, and optionally with one or more cytokines such as TNFα or IL-1β. Particularly preferred compositions for use in accordance with this aspect of the invention include, for example, compositions comprising: (a) an effective amount of each of Compound V and TNFα; (b) an effective amount of each of SR11237 and TNFα; or (c) an effective amount of each of SR 11237, Compound II and TNFα; or pharmaceutically acceptable salts thereof. As used herein, a compound or composition is said to “activate” an antigen-presenting cell if it alters or otherwise modulates (positively or negatively) one or more cellular phenotypes or physiological processes in the cell relative to the level of those particular cellular phenotypes or physiological processes in an antigen-presenting cell that has not been contacted by the compound or composition. Activation of an antigen-presenting cell may be determined according to any of a number of assays for cellular activation, including those described in detail herein and others that will be familiar to one of ordinary skill in the art in view of the teachings contained herein. According to this aspect of the invention, the antigen-presenting cell may be contacted with the one or more retinoids and one or more cytokines simultaneously or sequentially, in any order, in vivo, in vitro, or ex vivo, in amounts designed to activate the antigen-presenting cell.

In a related aspect, the invention provides methods of screening candidate compounds or compositions to select a compound or composition capable of activating an antigen-presenting cell. Preferred such methods may comprise, for example:

(a) obtaining a first and a second mammalian antigen-presenting cells;

(b) contacting the first cell, but not the second cell, with one or more compositions to be assayed for the ability to activate an antigen-presenting cell;

(c) determining the level of expression of a cellular phenotype associated with activation in the first and second cells;

(d) comparing the level of the cellular phenotype in the first and second cells; and

(e) selecting a composition wherein the level of the cellular phenotype in said first antigen-presenting cell is different from the level of the cellular phenotype in the second antigen-presenting cell, wherein a difference in the phenotype between the first and second cells is indicative of induction of activation in the first cell.

In another aspect, the invention provides additional methods of modulating the immune system of an animal (preferably a mammal such as a human), by affecting the physiology of an antigen-presenting cell. Preferred such methods may comprise, for example, inhibiting, delaying or preventing apoptosis of an antigen-presenting cell, particularly retinoid-induced apoptosis of an antigen-presenting cell. Such methods of the invention may comprise, for example, contacting the antigen-presenting cell with one or more retinoids and one or more cytokines, or with one or more of the compositions of the invention described hereinbelow, in amounts effective to inhibit, delay or prevent the apoptosis (particularly retinoid-induced apoptosis) of an antigen-presenting cell. Preferred methods and compositions for use in accordance with this aspect of the invention include contacting the cell with at least one RAR agonist (particularly at least one RARα agonist such as Compound I or a pharmaceutically acceptable salt thereof), at least one RAR antagonist (particularly an RARα antagonist such as Compound II or a pharmaceutically acceptable salt thereof, or a pan-RAR antagonist such as Compound V or Compound VIII, or pharmaceutically acceptable salts thereof), or at least one RXR agonist (particularly SR11237 or Compound V, or pharmaceutically acceptable salts thereof). Additional such embodiments may optionally further comprise contacting the antigen-presenting cell with an effective amount of one or more cytokines such as TNFα or IL-162 . Particularly preferred compositions for use in accordance with this aspect of the invention include, for example, compositions comprising: (a) an effective amount of Compound II; (b) an effective amount of each of Compound II and SR11237; (c) an effective amount of Compound V; (d) an effective amount of each of Compound V and SR11237; (e) an effective amount of Compound VIII; (f) an effective amount of each of Compound VIII and SR11237; or (g) an effective amount of each of TNFα and SR11237; or pharmaceutically acceptable salts thereof. As used herein, a compound or composition is said to “inhibit, delay or prevent” apoptosis in an antigen-presenting cell if it causes a decrease in, or delays the onset of, one or more physiological processes or characteristics in the cell that are associated with apoptosis, relative to the level of those particular physiological processes or characteristics in an antigen-presenting cell that has not been contacted by the compound or composition. For example, apoptosis of an antigen-presenting cell may be indicated by any phenotype, marker or activity that may be observed to change (i.e., increase or decrease) in expression, level or amount in an antigen-presenting cell that is undergoing apoptosis compared to an antigen-presenting cell that is not undergoing apoptosis. Apoptosis of an antigen-presenting cell may be determined according to any of a number of assays for cellular apoptosis, including those described in detail herein and others that will be familiar to one of ordinary skill in the art in view of the teachings contained herein. According to this aspect of the invention, the antigen-presenting cell may be contacted with the one or more retinoids and one or more cytokines simultaneously or sequentially, in any order, in vivo, in vitro, or ex vivo, in amounts designed to inhibit, delay or prevent apoptosis in the antigen- presenting cell.

In related aspects, the invention provides methods for screening candidate compounds in order to identify compounds capable of inhibiting, delaying or preventing the apoptosis of an antigen-presenting cell. One such method may comprise, for example:

(a) contacting a first and a second antigen-presenting cell with one or more compounds that induce apoptosis in the antigen-presenting cell (preferably one or more apoptosis-inducing retinoids such as one or more RARα agonists (e.g., tRA, 9cRA, or Compound I (see below), optionally in conjunction with one or more RXR agonists (e.g., SR11237, a pan-RXR agonist)), under conditions favoring the development of one or more cellular characteristics of apoptosis in the cell;

(b) contacting the first, but not the second, cell with one or more test compounds to be screened for the ability to inhibit, delay or prevent apoptosis in the first cell; and

(c) measuring the levels of one or more cellular characteristics of the apoptotic phenotype in the first and second cells, wherein a reduction in the level of such characteristics in the first cell relative to those in the second cell indicates that the test compound is able to inhibit, delay or prevent apoptosis in an antigen-presenting cell.

In another aspect, the invention provides additional methods of modulating the immune system of an animal (preferably a mammal such as a human), by affecting the physiology of an antigen-presenting cell. Methods according to this aspect of the invention may comprise, for example, inducing apoptosis of an antigen-presenting cell. Preferred such methods of the invention may comprise, for example, contacting the antigen-presenting cell with one or more apoptosis-inducing retinoids (which may preferably be one or more synthetic retinoids), or with one or more of the compositions of the invention described hereinbelow, in amounts effective to induce apoptosis in an antigen-presenting cell. Preferred methods and compositions for use in accordance with this aspect of the invention include contacting the cell with at least one RARα agonist (such as Compound I or a pharmaceutically acceptable salt thereof), at least one RARβ agonist (such as Compound III or Compound VII, or pharmaceutically acceptable salts thereof), or at least one pan-RXR agonist (such as SR11237 or Compound V, or pharmaceutically acceptable salts thereof). Particularly preferred compositions for use in accordance with this aspect of the invention include, for example, compositions comprising: (a) an effective amount of Compound I; (b) an effective amount of each of Compound I and SR11237; (c) an effective amount of Compound III; (d) an effective amount of each of Compound III and SR11237; (e) an effective amount of Compound IV; (f) an effective amount of each of Compound IV and SR11237; (g) an effective amount of Compound VII; or (h) an effective amount of each of Compound VII and SR11237; or pharmaceutically acceptable salts thereof. As used herein, a compound or composition is said to “induce” apoptosis in an antigen-presenting cell if it causes an increase in, or accelerates the onset of, one or more physiological processes or characteristics in the cell that are associated with apoptosis, relative to the level of those particular physiological processes or characteristics in an antigen-presenting cell that has not been contacted by the compound or composition. For example, apoptosis of an antigen-presenting cell may be indicated by any phenotype, marker or activity that may be observed to change (i.e., increase or decrease) in expression, level or amount in an antigen-presenting cell that is undergoing apoptosis compared to an antigen-presenting cell that is not undergoing apoptosis. Such phenotypes, markers or activities may include, but are not limited to, those described above and in the Examples below.

In additional related aspects, the invention provides methods for screening candidate compounds in order to identify compounds capable of inducing the apoptosis of an antigen-presenting cell. One such method may, for example, comprise:

(a) obtaining a first and a second antigen-presenting cell;

(b) contacting the first, but not the second, cell with one or more test compounds to be screened for the ability to induce apoptosis in the first cell; and

(c) measuring the levels of one or more cellular characteristics of the apoptotic phenotype in the first and second cells, wherein an increase in the level of such characteristics in the first cell relative to those in the second cell indicates that the test compound is able to induce apoptosis in an antigen-presenting cell.

The invention also relates to methods of treating an animal (preferably a mammal and most preferably a human) suffering from, susceptible to, or predisposed to a physical disorder, comprising administering to the mammal an effective amount of at least one retinoid and optionally at least one cytokine, which may be administered to the animal in the form of one or more of the compositions of the present invention. These methods of the invention are useful in treating or preventing a variety of physical disorders in an animal, including but not limited to infectious diseases, cancers, immune system disorders and the like, as described in detail herein.

### Retinoids

As noted above, the term “retinoid” as used herein is a compound which binds to one or more of the retinoid receptors (RARα, RARβ, RARγ, RXRα, RXRβ and RXRγ). Compounds are either “RAR retinoids” or “RXR retinoids” depending on their binding characteristics (RAR retinoids bind to one or more RARs; RXR retinoids bind to one or more RXRs (also referred to as “rexinoids”)). Retinoids which cause or otherwise positively modulate transactivation via their receptors are examples of “agonists,” while retinoids which do not cause transactivation, but instead block or otherwise negatively modulate the transactivation caused by other agonists, are examples of “antagonists.” RXR and RAR agonists to be used in the methods and compositions of the present invention can be, but are not limited to, peptides, carbohydrates, steroids and vitamin derivatives, which may each be natural or synthetic (prepared, for example, using methods of synthetic organic and inorganic chemistry that are well-known in the art).

By retinoids that are “specific” for a retinoid receptor are intended compounds that only bind to a particular retinoid receptor. By retinoids that are “selective” for a retinoid receptor are intended compounds that preferably bind to a particular retinoid receptor over others by a magnitude of approximately five-fold or greater than to other retinoid receptors, preferably eight-fold or greater, more preferably, ten-fold or greater.

Standard retinoids known in the art as RAR agonists include the following:

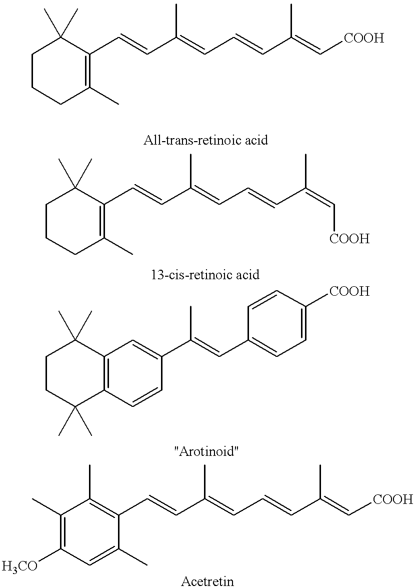

RARα,β-selective agonists include, but are not limited to,

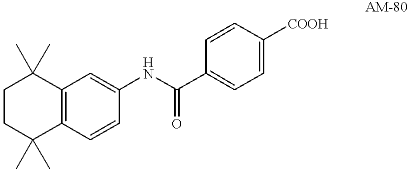

(see, Takeuchi, M., et al., Brit. J. Haematol. 97:137-140 (1997)).

RARβ,γ-selective agonists include, but are not limited to,

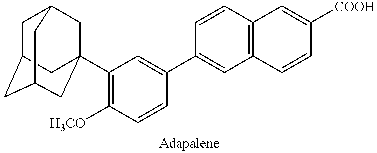

(see, Shroot, B. and Michel, S., J. Amer. Acad. Dermatol. 36:S96-S 103 (1997)).

RARγ agonists include, but are not limited to,

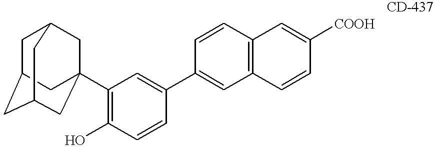

(see, Schadendorf, D., et al., Intl. J. Oncol. 5:1325-1331 (1994)); and

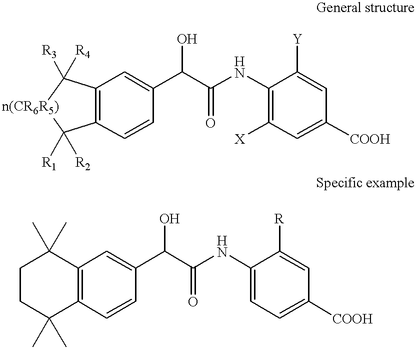

particularly wherein in the above-noted general structure R₁-R₆ are H or alkyl; n is 1-4; X is F, Cl, OH, or CH₃; Y is H or F; and wherein in the above-noted specific example, R is the same as X. (see, Swann, R. T., et al., EP 0 747 347).

RAR agonists include, but are not limited to,

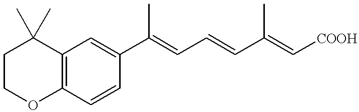

(see, Benbrook, D. M., et al., J. Med. Chem. 40:3567-3583 (1997));

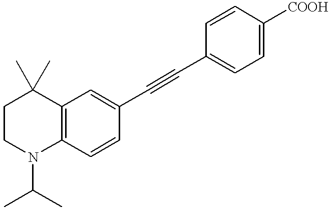

(see, Beard, R. L., et al., Bioorg. Med. Chem. Lett. 7:2372-2378 (1997)); and

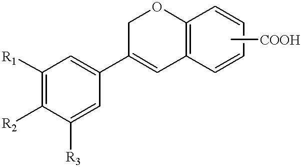

(see, Diaz, P., et al., Bioorg. Med. Chem. Lett. 7:2289-2294 (1997)), particularly wherein R₁ is adamantyl or t-butyl, R₂ is OH or OCH₃, R₃ is H or t-butyl, or R₁ and R₂ taken together form a 6-membered ring optionally substituted with up to four CH₃ groups

Further, RARα specific or selective agonists can contain an amide group. RARγ specific or selective agonists can contain a hydroxyl group or a carbonyl group such as a flavone structure. RARβ specific or selective agonists can be characterized by the absence of a hydroxy and amide groups. Moreover, it has been determined that RARβ specific agonists can be characterized by a dihydronaphthalene nucleus bearing a 2-thienyl group at C8 (see, U.S. Pat. No. 5,559,248; Johnson, A. T., et al., J. Med. Chem. 39:5029-5030 (1996)).

General RXR agonists include, but are not limited to,

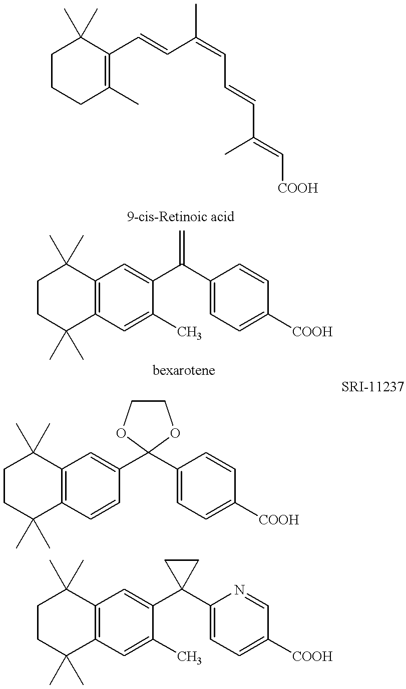

Additional RXR agonists include, but are not limited to,

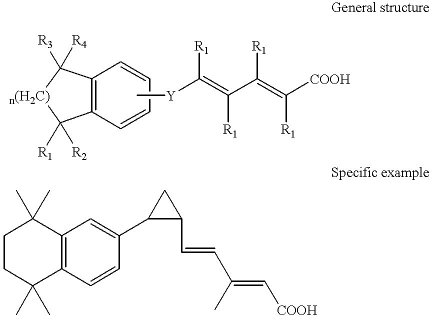

(see, Vuligonda, V. And R. A. Chandraratna, U.S. Pat. No. 5,675,033), particularly wherein in the above-noted general structure, R₁-R₄ are independently H, alkyl, or fluoroalkyl; Y is cycloalkyl or cycloalkenyl of 3-8 carbon atoms or is selected from phenyl, pyridyl, thienyl, furyl, pyrrolyl, pyridazinyl, pyrimidinyl, pyrazinyl, thiazolyl, oxazolyl, and imidazolyl, any of which can be substituted with 1-2 R₄ groups, the divalent Y radical being substituted by the phenyl ring and the alkene on adjacent carbon atoms; and n is 1 or 2;

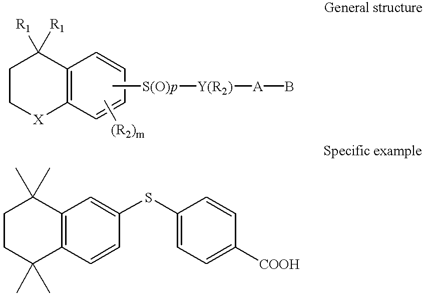

(see, Beard, R. L., et al., WO 97/16422), particularly wherein in the above-noted general structure, X is S, O or NR′, where R′ is H or alkyl of 1-6 carbons, or X is [C(R₁)₂]ₙ, where n is an integer between 0 and 2; R₁ is independently H or alkyl of 1-6 carbons; R₂ is independently H or alkyl of 1-6 carbons, halogen, fluoro-substituted alkyl of 1-6 carbons, OR″ or SR″ where R″ is H or alkyl of 1-6 carbons; m is an integer from 0-3; p is an integer from 0-2; Y is a phenyl or naphthyl group or heteroaryl selected from the group consisting of pyridyl, thienyl, furyl, pyridazinyl, pyrimidinyl, pyrazinyl, thiazolyl, oxazolyl, imidazolyl, and pyrazolyl, said phenyl and heteroaryl groups being optionally substituted with one or two R₂ groups; A is (CH₂)_{q}, where q is 0-5, lower branched chain alkyl having 3-6 carbons, cycloalkyl having 3-6 carbons, alkenyl having 2-6 carbons and 1 or 2 double bonds, alkynyl having 2-6 carbons and 1 or 2 triple bonds; and B is hydrogen, COOH or a pharmaceutically acceptable salt thereof, COOR₈, RONR₉R₁₀, CH₂OH, CH₂OR₁₁, CHO, C(OR₁₂)₂, CHOR₁₃O, COR₇, CR₇(OR₁₂)₂, CR₇OR₁₃O or tri-lower alkylsilyl, where R₇ is an alkyl, cycloalkyl or alkenyl group containing 1 to 5 carbons, R₈ is an alkyl group of 1 to 10 carbons or (trimethylsilyl)alkyl, where the alkyl group has 1 to 10 carbons, or a cycloalkyl group of 5 to 10 carbons, or R₈ is phenyl or lower alkylphenyl, R₉ and R₁₀ independently are hydrogen, an alkyl group of 1 to 10 carbons or a cycloalyl group of 5-10 carbons, or phenyl or lower alkylphenyl, R₁₁ is lower alkyl, phenyl or lower alkylphenyl R₁₂ is lower alkyl, and R₁₃ is a divalent alkyl radical of 2-5 carbons;

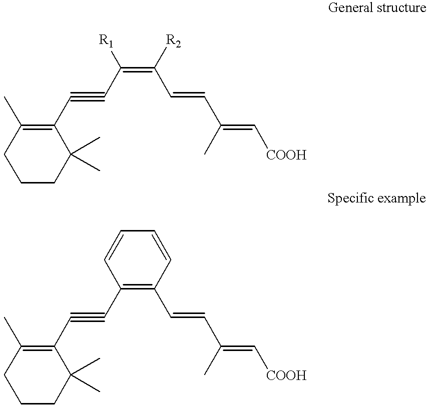

(see, Klaus, M., et al., EP 0 728 742), particularly wherein in the above-noted general structure, R₁ and R₂ taken together are C₃₋₁₃ alkylene in which one carbon atom may be substituted by a heteroatom selected from the group consisting of S, O and N, or R₁ and R₂ taken together with the carbon atoms to which they are attached are an aromatic ring having from 5-6 carbon atoms or a heteroaromatic ring having from 5-6 atoms in which one atom of R₁ or R₂ is a heteroatom selected from the group consisting of N, O and S and the remaining atoms of R₁ and R₂ are carbon;

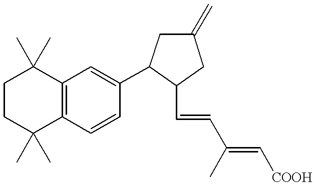

(see, Farmer, L. J., et al., Bioorg. Med. Chem. Lett. 7:2393-2398 (1997)); and

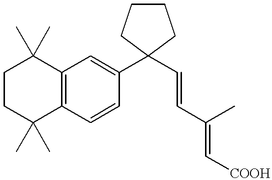

(see, Farmer, L. J., et al., Bioorg. Med. Chem. Lett. 7:2747-2752 (1997)).

RAR or RXR agonists include, but are not limited to,

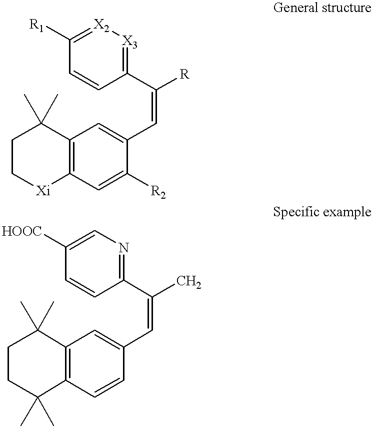

(Leblond, B., WO 97/26237), particularly wherein in the above-noted general structure, X₁ is C, which may be substituted with 1-2 CH₃ groups, O, or S; X₂ and X₃ are independently C(—H), O, N, or S, or taken together may be a single O, N, or S atom; R₁ may be H, lower alkyl, OH, CH₂OH, CHO, COOH, COO-alkyl, or tetrazole, the tetrazole may be further optionally substituted with a CF₃ group; R₂ may be H, lower alkyl, halo-substituted alkyl, halogen, OH or O-alkyl; and R may be H, lower alkyl, or halo-substituted alkyl.

Other RXR agonists, with a variety of structures, are disclosed in Boehm, M. F., et al., J. Med. Chem. 38:3146-3155 (1995). Further, a number of retinoids of diverse structure types which are triple RAR agonists, selective RARα agonists, selective RARβ agonists, selective RARγ agonists, selective RARβ,γ agonists, selective RXR agonists and RXR/RAR pan-agonists are described in Sun, S. Y., et al., Cancer Res. 57:4931-4939 (1997). The invention can also be carried out with the RXR agonist bexarotene, the structure and preparation of which are described in Boehm et al., J. Med. Chem. 37:2930-2941 (1994). Other RXR agonists are also described in, for example, Lehmann et al., Science 258:1944-1946 (1992), and in commonly owned, co-pending U.S. Appl. Ser. Nos. 08/919,318, filed Aug. 28, 1997, 09/065,904, filed Apr. 24, 1998, and 60/130,649, filed Apr. 23, 1999, the entire disclosures of which are incorporated herein by reference.

Other candidate RAR and/or RXR agonists include, but are not limited to,

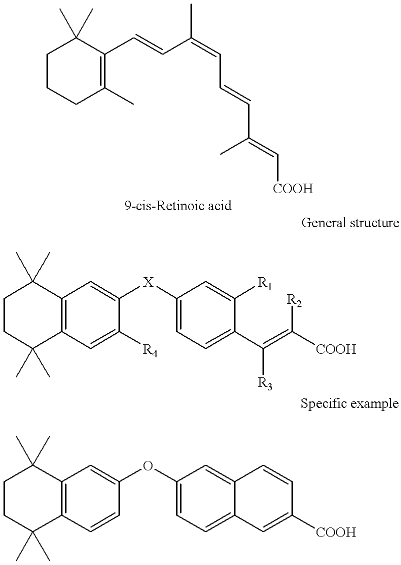

(see, Bernardon, J. M., EP 0 722 928), particularly wherein in the above-noted general structure, X is O, S(O)ₙ or N-R₅, where n is an integer from 0-2 and R₅ is H or lower alkyl; R₁ is H, alkyl, OH, O-alkyl or O—C(═O)-alkyl; R₂ may be H, alkyl, OH, O-alkyl, O—C(═O)-alkyl, NH₂, or NH—C(═O)-alkyl; R₃ may be H or alkyl; or R₂ and R₃ taken together may form a bond; R₄ may be H, lower alkyl, halogen, NO₂, OH, O-alkyl, NR₅R₆, where R₅ has the definition above and R₆ may be independently H or lower alkyl;

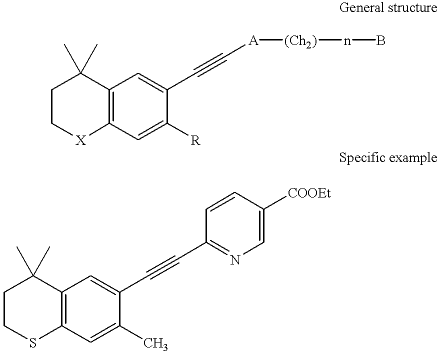

(see, Chandraratna, R., WO 96/11686; and Drugs of the Future 22:249-255 (1997)), particularly wherein in the above-noted general structure, X is S, O, or NR′, where R′ is H or lower alkyl; R is H or lower alkyl, A is pyridyl, thienyl, furyl, pyridazinyl, pyrimidinyl or pyrazinyl; n is 0-4, and B is H, COOH or a pharmaceutically acceptable salt, ester, or amid thereof, or CH₂OH;

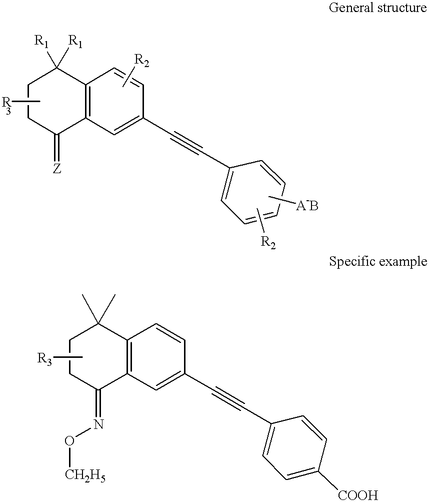

(see, Vuligonda, S., et al., U.S. Pat. No. 5,599,967), particularly wherein in the above-noted general structure and specific example, R₁ is H or lower alkyl, R₂ and R₃ are independently H or lower alkyl; A is (CH₂)ₙ, where n is an integer from 0-5, lower branched chain alkyl having 3-6 carbons, cycloalkyl having 3-6 carbons, alkenyl having 2-6 carbons and 1-2 double bonds or alkynyl having 2-6 carbons and 1-2 triple bonds; B is H, COOH or a pharmaceutically acceptable salt, ester, or amide thereof, or CH₂OH; and Z is N—O—X, where X is alkyl, phenyl, benzyl, substituted phenyl, substituted benzyl, acyl, silyl, aryl or heteroaryl;

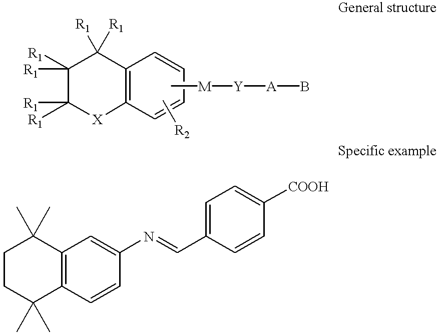

(see, Chandraratna, R. A. and M. Teng, WO 96/06070), particularly wherein in the above-noted general structure, the R₁ groups are independently H, lower alkyl of 1-6 carbons, or two geminal R₁ groups jointly represent an oxo (═O) or thio (═S) group; R₂ is H, lower alkyl, or halogen; M is —N═CR₄— or —CR₄═N—, where R₄ is H or lower alkyl; X is C(R₁)₂, O, S, or NR₁; Y is a (substituted) phenyl or heteroaryl group selected from pyridyl, thienyl, furyl, pyridazinyl, pyrimidinyl, pyrazinyl, thiazolyl, imidazolyl, and oxazolyl; A is (CH₂)ₙ, where n is an integer from 0-5, lower branched chain alkyl having 3-6 carbons, cycloalkyl having 3-6 carbons, alkenyl having 2-6 carbons and 1-2 double bonds or alkynyl having 2-6 carbons and 1-2 triple bonds; B is H, COOH or a pharmaceutically acceptable salt, ester, or amid thereof, or CH₂OH;

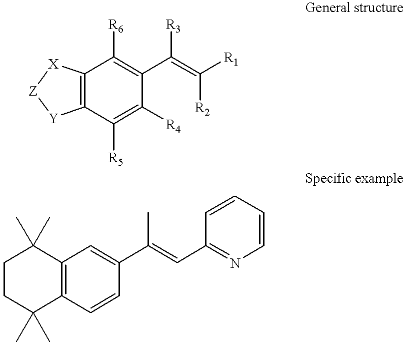

(see, Klaus, M. and E. Weis, EP 0 253 302), particularly wherein in the above general structure, X and Y are CH₂ or C(CH₃)₂; Z is CHR₈, C═O, CR₈OR₇, CHR₈—CHR₈, CHOR₇—CH₂, CO—CHOR₇, or CHOR₇—CHOR₇; R₁ is a 5- or 6-membered heterocyclic ring which can be further substituted with halogen, alkyl, alkoxy, acyloxy, nitro, hydroxy, amino, lower-alkylamino or di-lower-alkylamino groups; R₂ and R₃ may be H, lower alkyl, trifluoromethyl or halogen; R₄ and R₅ may be H, lower allyl or halogen, R₆ may be H, lower alkyl, or alkoxy, R₇ may be H, lower alkyl, or acyl; and R₈ may be H or lower alkyl;

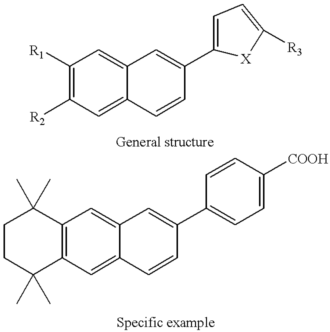

(see, Shroot, B. V., et al., EP 0210929), particularly wherein in the above general structure, X represents —CHCH—, O, or S; R₁ and R₂ may independently represent H, lower alkyl, alkoxy, or R₁ and R₂ taken together may be a 5- or 6-membered carbocyclic ring optionally saturated and optionally substituted by up to 5 lower alkyl groups; R₄is CH₂OH, COOH, or pharmaceutically acceptable salts, amides, or esters thereof; and

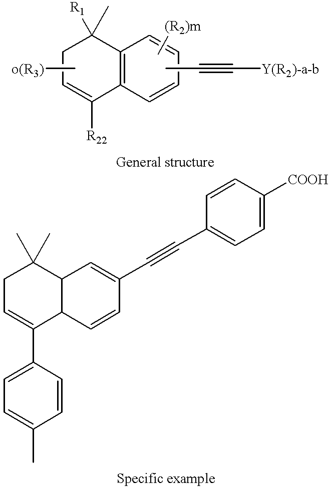

(see, Johnson, A. T., et al., U.S. Pat. No. 5,648,514), particularly wherein in the above general structure, R₁ is H or lower alkyl, R₂ and R₃ are H or lower alkyl, and the ethynyl group occupies either the 2- or 3- position of the dihydronaphthalene nucleus; m and o are integers from 0-3; Y is a phenyl group or heterocycle selected from the group consisting of pyridyl, thienyl, furyl, pyridazinyl, pyrimidinyl, pyrazinyl, thiazolyl, oxazolyl and imidazolyl, said groups being optionally substituted with 1-2 R₂ groups; a is (CH₂)ₙ, where n is an integer from 0-5, lower branched chain alkyl having 3-6 carbons, cycloalkyl having 3-6 carbons, alkenyl having 2-6 carbons and 1-2 double bonds or alkynyl having 2-6 carbons and 1-2 triple bonds; b is H, COOH or a pharmaceutically acceptable salt, ester, or amide thereof, or CH₂OH; R₂₂ is H, lower alkyl, fluoro-substituted alkyl of 1-10 carbons, alkenyl of 2-10 carbons containing 1-3 double bonds, alkynyl having 2-10 carbons and 1-3 triple bonds, carbocyclic aryl selected from the group consisting of phenyl, C₁-C₁₀ alkylphenyl, naphthyl, C₁-C₁₀ alkylnaphthyl, phenyl-C₁-C₁₀ alkenyl having 1-3 double bonds, phenyl-alkynyl having 1-3 triple bonds, hydroxyalkyl, acyloxyalkyl, aminoalkyl, thioalkyl, or a 5-6 membered heteroaryl group optionally substituted with a C₁-C₁₀ alkyl group, said heteroaryl group having 1-3 heteroatoms selected from O, N and S.

Thus, preferred retinoids that can be used in accordance with the present invention include, but are not limited to, one or more RARα agonists such as 4-[[(2,3-Dihydro-1,1,3,3-tetramethyl-2-oxo-1H-inden-5-yl)carbonyl]amino]-benzoic acid (referred to herein as “Compound I” and the structure and synthesis of which is shown in WO 98/47861, which is incorporated herein by reference), one or more RXR agonists (particularly one or more pan-RXR agonists such as SR11237 (also known as SRI1237, and the structure and synthesis of which are disclosed in U.S. Pat. Nos. 5,466,861 and 5,552,271 which are incorporated herein by reference) and/or 4-[1-[5,6-Dihydro-3,5,5-trimethyl-8-(1-methylethyl)-2-naphthalenyl]ethenyl]-benzoic acid (referred to herein as “Compound V” and the structure and synthesis of which are provided in commonly owned, co-pending U.S. Appl. Ser. Nos. 60/127,976, filed Apr. 6, 1999, and 60/130,649, filed Apr. 23, 1999, which are incorporated herein by reference), one or more RAR antagonists (particularly one or more RARα antagonists such as 4-[[[5,6-Dihydro-5,5-dimethyl-8-(3-quinolinyl)-2-naphthalenyl]carbonyl]-amino]benzoic acid (referred to herein as “Compound II” and the structure and synthesis of which are disclosed in U.S. Pat. Nos. 5,559,248 and 5,849,923, which are incorporated herein by reference), or one or more pan-RAR antagonists such as 4-[(1E)-2-[5,6-dihydro-5,5-dimethyl-8-phenylethynyl)-2-naphthalenyl]ethenyl]benzoic acid (referred to herein as “Compound VIII” and the structure and synthesis of which are disclosed in WO 98/46288, which is incorporated herein by reference)).

An additional preferred retinoid for use in accordance with the invention is (E)-3-chloro-4-[2-(5,6-dihydro-5,5-dimethyl-8-phenyl-2-naphthalenyl)-ethenyl]benzoic acid (referred to herein as “Compound III”). Compound III can be synthesized by using the following intermediate (X═Cl):

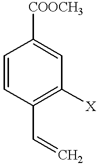

The above intermediate can be synthesized from the commercially available 3-chloro-4-hydroxybenzoic acid as shown below:

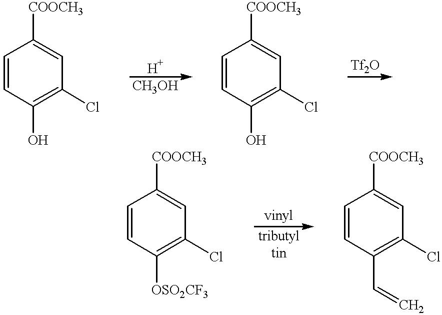

In this synthesis, the acid is first esterified, followed by activation of the OH group with trifluoromethane sulfonic anhydride (Tf₂O), then the trifluoromethane sulfonate group is coupled with vinyl tributyl tin to give the desired intermediate, which is then used to prepare Compound III as described in U.S. Pat. No. 5,618,839, which is incorporated by reference herein.

For sake of brevity, the following designations for non-limiting examples of synthetic retinoids useful in the present invention are used throughout this disclosure:

TABLE 1A
Non-limiting Examples of Synthetic Retinoids.

| Compound | Structure | CAS Name |
|---|---|---|
| I | 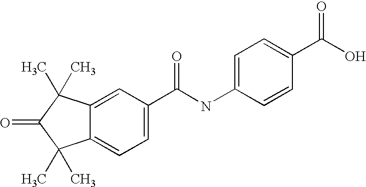 | 4-[[(2,3-Dihydro-1,1,3,3- tetramethyl-2-oxo-1H-inden- 5-yl)carbonyl]amino]benzoic acid |
| II | 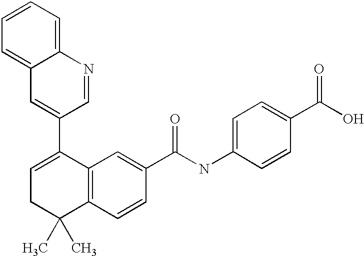 | 4-[[[5,6-Dihydro-5,5- dimethyl-8-(3-quinolinyl)-2- naphthalenyl]carbonyl]amino]benzoic acid |
| III | 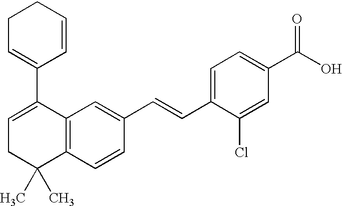 | (E)-3-Chloro-4-[2-(5,6- dihydro-5,5-dimethyl-8- phenyl-2-naphthalenyl)- ethenyl] benzoic acid |
| IV | 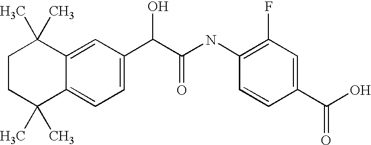 | 3-Fluoro-4[[(5,6,7,8- tetrahydro-5,5,8,8- tetramethyl-2- naphthalenyl)hydroxyacetyl]amino]benzoic acid |
| V | 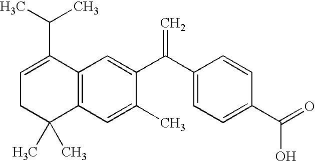 | 4-[1-[5,6-Dihydro-3,5,5- trimethyl-8-(1-methylethyl)- 2-naphthalenyl]ethenyl]benzoic acid |
| VI | 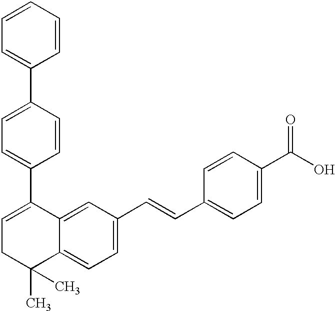 | (E)-4-[2-[8-(1,1′-Biphenyl]- 4-yl)-5,6-dihydro-5,5- dimethyl-2-naphthalenyl]- ethenyl]benzoic acid |
| VII | 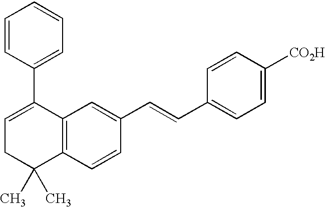 | 4-[(E)-2[5,6-dihydro-5,5- dimethyl-8-phenyl)-2- naphthalenyl]ethenyl]benzoic acid |
| VIII | 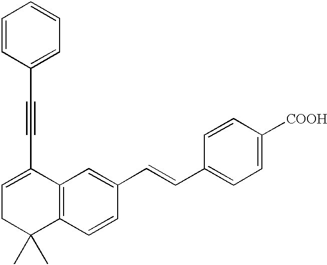 | 4-[(E)-2-[5,6-dihydro-5,5- dimethyl-8-phenylethynyl)-2- naphthalenyl]ethenyl]benzoic acid |
| SR 11237 | 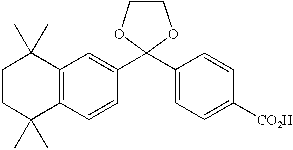 | 4-[2-(5,6,7,8-tetrahydro- 5,5,8,8-tetramethyl-2- naphthalenyl)-1,3-dioxolan- 2-yl]benzoic acid |

Structures and/or methods of synthesis of these compounds, and others suitable for use in accordance with the present invention, are known from the following patents, applications and publications, all of which are incorporated by reference herein:

TABLE 1b
References for Non-limiting Examples of Synthetic Retinoids

| Compound | Reference(s) |
|---|---|
| I | WO 98/47861 |
| II | U.S. Pat. No. 5,849,923 |
| III | EP 0 661 259; U.S. Pat. No. 5,618,839; U.S. Appl. |
| | No. 60/130,649, filed April 23, 1999 |
| IV | EP 0 747 347; U.S. Pat. No. 5,624,957 |
| V | U.S. Appl. Nos. 60/127,976, filed April 6, 1999, and |
| | 60/130,649, filed April 23, 1999 |
| VI | WO 98/46228 |
| VII | EP 0 661 259; U.S. Pat. No. 5,618,839 |
| VIII | WO 98/46228 |
| SR11237 | U.S. Pat. Nos. 5,466,861 and 5,552,271 |

Other retinoids suitable for use in accordance with the present invention may be prepared by the methods described or referenced herein, and by routine methods of organic and inorganic synthetic chemistry that will be familiar to those of ordinary skill in the art in view of the disclosure herein. For example, also intended to be encompassed within the methods and compositions of the present invention are the salt, ester, and prodrug forms of the specific compounds disclosed herein and others that will be known to the ordinarily skilled artisan in view of the present disclosure and information available in the art.

### Screening of Compositions

In another preferred aspect, the invention provides methods of identifying or selecting compositions or compounds capable of activating an antigen-presenting cell. Preferred methods according to this aspect of the invention may comprise one or more steps, which may be, in some assays, performed in any particular sequence provided that the protocols used provide appropriate and usable information regarding the activity of the screened compositions. For example, one such method according to the invention may comprise:

(a) obtaining a first and a second mammalian antigen-presenting cells;

(b) contacting said first cell, but not said second cell, with one or more compositions to be assayed for the ability to activate an antigen-presenting cell;

(c) determining the level of expression of a cellular phenotype associated with activation in said first and second cells;

(d) comparing the level of said cellular phenotype in said first and second cells; and

(e) selecting a composition wherein the level of said cellular phenotype in said first antigen-presenting cell is different from the level of said cellular phenotype in said second antigen-presenting cell, wherein a difference in the phenotype between said first and second cells is indicative of induction of activation in said first cell.

In additional related aspects, the invention provides methods for screening candidate compounds in order to identify compounds capable of inhibiting, delaying or preventing the apoptosis of an antigen-presenting cell. Preferred methods according to this aspect of the invention may also comprise one or more steps, which in some assays may be performed in any particular sequence provided that the protocols used provide appropriate and usable information regarding the activity of the screened compositions. For example, one such method according to the invention may comprise:

(a) contacting a first and a second antigen-presenting cell with one or more compounds that induce apoptosis in the antigen-presenting cell (preferably one or more apoptosis-inducing retinoids such as one or more RARα agonists (e.g., tRA, 9cRA, or Compound I (see below), optionally in conjunction with one or more RXR agonists (e.g., SR11237, a pan-RXR agonist)), under conditions favoring the development of one or more cellular characteristics of apoptosis in the cell;

(b) contacting the first, but not the second, cell with one or more test compounds to be screened for the ability to inhibit, delay or prevent apoptosis in the first cell; and

(c) measuring the levels of one or more cellular characteristics of the apoptotic phenotype in the first and second cells, wherein a reduction in the level of such characteristics in the first cell relative to those in the second cell indicates that the test compound is able to inhibit, delay or prevent apoptosis in an antigen-presenting cell.

In analogous related aspects, the invention provides methods for screening candidate compounds in order to identify compounds capable of inducing apoptosis in an antigen-presenting cell. Preferred methods according to this aspect of the invention may also comprise one or more steps, which may be, in some assays, performed in any particular sequence provided that the protocols used provide appropriate and usable information regarding the activity of the screened compositions. For example, one such method according to the invention may comprise:

(a) obtaining a first and a second antigen-presenting cell;

(b) contacting the first, but not the second, cell with one or more test compounds to be screened for the ability to induce apoptosis in the first cell; and

(c) measuring the levels of one or more cellular characteristics of the apoptotic phenotype in the first and second cells, wherein an increase in the level of such characteristics in the first cell relative to those in the second cell indicates that the test compound is able to induce apoptosis in an antigen-presenting cell.

### Screening Methods

The first and second antigen-presenting cells used in the present selection methods may be normal cells, immature cells, mature cells, diseased cells, transformed cells or established cell lines, and preferably are mammalian (particularly human) antigen-presenting cells. According to the invention, the first and second antigen-presenting cells are preferably of the same type and undergo the same incubation conditions, except that the first cell is contacted with one or more of the compositions or compounds to be assayed for its ability to activate an antigen-presenting cell, while the second cell is incubated in parallel with the first cell but in the absence of the one or more compositions or compounds to be assayed. Thus, the second cell serves as a “control” cell to indicate the levels of expression of one or more cellular phenotypes that are typically seen in that particular cell type in the absence of the compositions or compounds to be assayed (sometimes referred to in the art as the “quiescent,” “resting” or “non-activated” state in assays for compounds that activate cells), and provides a reference for determining the abilities of the compositions or compounds to activate, or induce or inhibit/delay/prevent apoptosis in, antigen-presenting cells.

According to the invention, the cellular phenotype or physiological process associated with activation in the antigen-presenting cells may be any phenotype, marker or activity that may be observed to change (i.e., increase or decrease) in expression, level or amount in an activated antigen-presenting cell compared to an antigen-presenting cell that has not been activated. Such phenotypes, markers or activities may include, but are not limited to, antigen processing and cell surface presentation (see Examples 4, 5 and 8 herein), differential expression of cell surface markers or antigens (particularly CD antigens), alterations in the translocation and binding of KF-κB complexes to their particular DNA response elements (see Example 6 herein), alterations in DNA replication (measured, e.g., by increases in the amount of labeled nucleotides that are incorporated into cellular DNA in the presence of the compositions or compounds being assayed), and the like. Of course, other cellular phenotypes that may be associated with activation of antigen-presenting cells and that may therefore be used in accordance with the methods of the invention will be apparent to the ordinarily skilled artisan in view of the teachings herein, and hence are considered to be encompassed within the methods of the invention.

Similarly, according to the invention the cellular phenotype or physiological process associated with apoptosis in the antigen-presenting cells may be any phenotype, marker or activity that may be observed to change (i.e., increase or decrease) in expression, level or amount in an antigen-presenting cell that is undergoing apoptosis compared to an antigen-presenting cell that is not undergoing apoptosis. Such phenotypes, markers or activities may include, but are not limited to, DNA fragmentation (determination of which may be accomplished by methods such as gel electrophoresis (appearance of smaller molecular weight bands)), changes in microscopic phenotype (e.g., changes in plasma membrane morphology such as formation of surface protuberances (“blebbing”) or in nuclear morphology such as pycnosis or fragmentation), a decrease in the expression of the putative apoptosis suppressive protein BCL-2 (decreased in apoptotic cells), the appearance in a cell population of smaller cells with different light scatter and/or DNA content profiles as assessed, for example, by flow cytometry or particle analysis, and other methods that are well-known in the art (see, e.g., Nagy, L., et al., Mol. Cell. Biol. 15(7):3540-3551 (1995); Horn, V., et al., FASEB J. 10:1071-1077 (1996); Telford, W. G., et al., J. Immunol. Meth. 172(1):1-16 (1994); Campana, D., et al., Cytometry 18(2):68-74 (1994); Sgonc, R., and Wick, G., Int. Arch. Allergy Immunol. 105(4):327-332 (1994); Fraker, P. J., et al., Meth. Cell Biol. 46:57-76 (1995); Sherwood, S. W., and Schimke, R. T., Meth. Cell Biol. 46:77-97 (1995); Carbonari, M., et al., Cytometry 22(3):161-167 (1995); Mastrangelo, A. J., and Betenbaugh, M. J., Curr. Opin. Biotechnol. 6(2):198-202 (1995); and EP 0 903 149, the disclosures of all of which are incorporated herein by reference in their entireties).

As an alternative to using antigen-presenting cells, transfected cell lines may be constructed and used to preliminarily identify candidate retinoids that might be used in accordance with the compositions and methods of the invention. For example, in Chen et al., EMBO J. 14(6):1187-1197 (1995), three ‘reporter’ cell lines have been used to characterize a number of RARα-, RARβ-, or RARγ-specific dissociating synthetic retinoids that selectively induce the AF-2 activation function present in the ligand-binding domain (LBD) of RARβ (βAF-2). These cell lines stably express chimeric proteins containing the DNA binding domain of the yeast transactivator GAL4 fused to the EF regions (which contain that LBD and the AF-2 activation function) of RARα (GAL-RARα), RARβ (GAL-RARβ) or RARγ (GAL-RARγ), and a luciferase reporter gene driven by a pentamer of the GAL4 recognition sequence (“17 m”) in front of the β-globin promoter (17 m)5-GAL-Luc). In these cell lines, the retinoids thus induce luciferase activity that can be measured in the intact cells using a single-photon-counting camera. This reporter system is insensitive to endogenous receptors which cannot recognize the GAL4 binding site. Using analogous screening assays, synthetic retinoids have been reported to inhibit the anchorage-independent growth of oncogene-transformed 3T3 cells, while the promoter of the human interleukin-6 (IL-6) gene, whose product is involved in the regulation of hematopoiesis, immune responses and inflammation (Kishimoto, T. et al., Science 258:593-597 (1992)), has been shown to be induced by Rol but not by the synthetic dissociating retinoids which repressed its activity. In a similar manner, activating RXR agonists have been identified using cell lines that express a RXR receptor linked to a TREpal-tk reporter gene which is activated by both RAR-RXR heterodimers and RXR homodimers (Lehmann, J. M., et al., Science 258:1944-1946 (1992)). Thus, reporter cell lines that are easily constructed, by methods routine to one of ordinary skill, may be used to distinguish not only the specific RAR or RXR types to which a candidate ligand will bind, but also whether that binding induces an activating or inhibiting (i.e., agonistic or antagonistic) effect. Although the above-referenced reporter cell lines comprised the luciferase or thymidine kinase genes as reporters, other reporters such as Neo, CAT, β-galactosidase or Green Fluorescent Protein are well known in the art and may be used in a similar fashion to carry out the present invention. Other references disclosing reporter plasmids containing a reporter gene and expression vectors encoding a LBD of a nuclear receptor include Meyer et al., Cell 57:433-442 (1989); Meyer et al., EMBO J. 9(12):3923-3932 (1990); Tasset et al., Cell 62:1177-1187 (1990); Gronemeyer, H., and Laudet, V., Protein Profile 2:1173-1308 (1995); Webster et al., Cell 54:199-207 (1988); Strähle et al., EMBO J. 7:3389-3395 (1988); Seipel et al., EMBO J. 11:4961-4968 (1992); and Nagpal et al., EMBO J. 12:2349-2360 (1993).

Other routine assays have been used to screen compounds for their agonistic properties on functions of other nuclear receptors, such as steroid receptors. For example, a transient expression/gel retardation system has been used to study the effects of the synthetic steroids RU486 and R5020 on glucocorticoid and progesterone receptor function (Meyer, M -E., et al., EMBO J. 9(12): 3923-3932 (1990)). Similar assays have been used to show that tamoxifen competitively inhibits estradiol-induced ERAP160 binding to the estrogen receptor, suggesting a mechanism for its growth-inhibitory effects in breast cancer (Halachimi, S., et al., Science 264:1455-1458 (1994)). Since the RAR and RXR receptors are apparently structurally similar to other nuclear receptors such as the steroid receptors (as reviewed in Chambon, P., FASEB J. 10:940-954 (1996)), routine assays of this type may be useful in assessing compounds for their abilities to modulate gene expression through RARs or RXRs. As an alternative routine method, the effect of a candidate compound or composition on the binding of the ligand-dependent AF-2 modulator TIF1 to a RAR or RXR LBD can be studied using glutathione-S-transferase (GST) interaction assays by tagging the LBDs with GST as described in detail in Le Douarin et al., EMBO J 14:2020-2033 (1995).

In another screening assay, transgenic mice and cell lines that are altered in their expression of one or more RAR or RXR receptors may be made as described previously (Krezel, W., et al., Proc. Natl. Acad. Sci. USA 93(17):9010-9014 (1996)) and may be used to identify agonists of specific members of the RAR/RXR class of receptors using methods described previously (WO 94/26100). In such an assay, the agent which is to be tested will be incubated with one or more of the transgenic cell lines or mice or tissues derived therefrom. The level of binding of the agent is then determined, or the effect the agent has on development or gene expression is monitored, by techniques that are routine to those of ordinary skill. As used herein, the term “incubate” is defined as contacting the compound or agent under investigation with the appropriate cell or tissue, or administering the agent or compound to the appropriate mouse, via any one of the well-known routes of administration including enteral, intravenous, subcutaneous, and intramuscular.

Other assays, such as those described in detail below in the Examples, may also be used to predict the modulatory effects of RAR and RXR ligands on immune system function (particularly antigen-presenting cell activation and/or apoptosis) by determining the agonistic effects of these ligands on other targets. For example, certain agonistic retinoids will induce the association of endogenous PML/PML-RARα fusion protein with nuclear bodies in cells from APL patients (Dyck, J. A., et al., Cell 76:333-343 (1994); Weis, K., et al., Cell 76:345-356 (1994); Koken, M. H. M., et al., EMBO J. 13(5):1073-1083 (1994)) or in related established cell lines such as NB4 (Lanotte, M., et al., Blood 77(5):1080-1086 (1991)). These effects of RAR or RXR agonists or antagonists may be determined, for example, by various immunological techniques such as immunofluorescent or immunoelectron microscopy, using antibodies specific for PML, RAR and/or PML-RARα fusion proteins. RAR or RXR agonists may also be identified by their abilities to induce the in vitro differentiation (maturation) of certain established cell lines such as HL-60 myeloblastic leukemia cells (Nagy, L., et al., Mol. Cell. Biol. 15(7):3540-3551 (1995)), NB4 promyelocytic cells (Lanotte, M., et al., Blood 77(5):1080-1086 (1991), P19 or F9 embryonic carcinoma cells (Roy, B., et al., Mol. Cell. Biol. 15(12):6481-6487 (1995); Horn, V., et al., FASEB J. 10: 1071-1077 (1996)), or ras-transformed 3T3 cells (Chen et al., EMBO J. 14(6): 1187-1197 (1995)). Ligand-induced differentiation in these and other cell lines may be determined by assaying ligand-treated or -untreated cells for the expression of a variety of well-known markers of differentiation as generally described in the above references.

Similarly, the candidate compounds may be screened by measuring their abilities to induce or inhibit/delay/prevent apoptosis (programmed cell death) in, for example, HL-60 cells (Nagy, L., et al., Mol. Cell. Biol. 15:3540-3551 (1995)), P19 cells (Horn, V., et al., FASEB J. 10:1071-1077 (1996)), or cells (particularly myeloid cells) expressing Integrin-Associated Protein (IAP; see EP 0 903 149), or in accordance with the invention in one or more antigen-presenting cells, precursors thereof, or cell lines derived therefrom, as described herein. In particular, screening assays according to this aspect of the invention may be carried out using dendritic cells, Langerhans cells, macrophages, or antigen- presenting cells derived from peripheral blood monocytes according to methods that are known in the art (see, e.g., Geissman et al., J. Exp. Med. 197:961-966 (1998), Geissman et al., J. Immunol. 162:4567-4575 (1999), and the Materials and Methods section in the Examples herein). Apoptosis in such cells is typically assessed by measurement of ligand-induced DNA fragmentation, which is accomplished by methods such as gel electrophoresis (appearance of smaller molecular weight bands), microscopy (changes in plasma membrane morphology such as formation of surface protuberances (“blebbing”) orin nuclearmorphology such as pycnosis or fragmentation) or expression of the putative apoptosis suppressive protein BCL-2 (decreased in apoptotic cells); for general methods and discussions of these assays as they pertain to RAR and RXR biology, see Nagy, L., et al., Mol. Cell. Biol. 15(7):3540-355 1 (1995); Horn, V., et al., FASEB J. 10:1071-1077 (1996)). Other methods for assaying ligand-induced apoptosis in primary cells and established cell lines, such as flow cytometry or particle analysis (appearance of smaller particles with different light scatter and/or DNA content profiles), are well-known in the art (Telford, W. G., et al., J. Immunol. Meth. 172(1):1-16 (1994); Campana, D., et al., Cytometry 18(2):68-74 (1994); Sgonc, R., and Wick, G., Int. Arch. Allergy Immunol. 105(4):327-332 (1994); Fraker, P .J., et al., Meth. Cell Biol. 46:57-76 (1995); Sherwood, S. W., and Schimke, R. T., Meth. Cell Biol. 46:77-97 (1995); Carbonari, M., et al., Cytometry 22(3):161-167 (1995); Mastrangelo, A. J., and Betenbaugh, M. J., Curr. Opin. Biotechnol. 6(2):198-202 (1995); EP 0 903 149). Such methods are also described in detail in the Examples herein.

Other methods for determining the ability of a candidate compound or composition to differentially modulate immune system (particularly antigen-presenting cell) function and/or to induce or inhibit/delay/prevent apoptosis in mammalian antigen-presenting cells, which are routine in the art, may also be used in carrying out the present invention. In performing such assays, the skilled artisan will be able to determine which RAR or RXR receptor type an agent binds to, what specific receptor(s) are utilized by a given compound, whether the agent is an agonist or an antagonist of the given receptor(s), and whether the compound or combination of compounds are capable of differentially modulating antigen presentation and/or apoptosis in antigen-presenting cells.

According to the invention, for assays to determine whether a compound is capable of activating, or of inhibiting/delaying/preventing apoptosis in, an antigen-presenting cell, the first mammalian cell, but not the second mammalian cell, is contacted with the one or more compounds or compositions to be assayed. The levels of one or more activation- or apoptosis-associated physiological processes or cellular phenotypes are then determined in the first and second cells according to one or more of the above-described assays, and the levels in the first cell are then compared to those in the second (control) cell. A compound or composition is then selected as activating, or inhibiting/delaying/preventing apoptosis in, an antigen-presenting cell if the level of one or more physiological processes or cellular markers that changes upon cellular activation (e.g., antigen processing and presentation; DNA replication; nuclear transcription complex translocation and DNA binding; CD86 antigen expression) or apoptosis (e.g., ligand-induced DNA fragmentation, changes in microscopic morphology, appearance of smaller particles with different light scatter and/or DNA content profiles) is at least about 30% higher, more preferably at least about 50%, 55%, 60%, 65%, 70%, 75%, 80%, 85%, 90%, 95%, 100%, 200%, 300%, 400%, 500%, etc., higher, in the cell that has been contacted with the compound or composition than the level of the same physiological processes or cellular markers in the (control) cell that has not been contacted with the composition or compound to be assayed. Analogously, a compound or composition is selected as activating, or inhibiting/delaying/preventing apoptosis in, an antigen-presenting cell if the level of a physiological process or cellular marker that decreases upon cellular activation (e.g., expression of antigens associated with an immature phenotype in Langerhans cells, such as CDla, E-cadherin, CLA, and Lag) or apoptosis (e.g., expression of the putative apoptosis suppressive protein BCL-2) is at least about 30% lower, more preferably at least about 50%, 55%, 60%, 65%, 70%, 75%, 80%, 83%, 85%, 90%, 93%, 95%, 97%, 98%, 99% or 100%, lower, in the cell that has been contacted with the compound or composition than the level of the same physiological processes or cellular markers in the (control) cell that has not been contacted with the composition or compound to be assayed.

Similarly, for assays to determine whether a compound is capable of inducing apoptosis in an antigen-presenting cell, the first, but not the second, cell is contacted with one or more test compounds to be screened for the ability to induce apoptosis in the first cell. The levels of one or more apoptosis-associated physiological processes or cellular phenotypes are then determined in the first and second cells according to one or more of the above-described assays, and the levels in the first cell are then compared to those in the second (control) cell. A compound or composition is then selected as inducing apoptosis in an antigen-presenting cell if the level of one or more physiological processes or cellular markers that increases upon apoptosis (e.g., ligand-induced DNA fragmentation, changes in microscopic morphology, appearance of smaller particles with different light scatter and/or DNA content profiles) is at least about 30% higher, more preferably at least about 50%, 55%, 60%, 65%, 70%, 75%, 80%, 85%, 90%, 95%, 100%, 200%, 300%, 400%, 500%, etc., higher, in the cell that has been contacted with the compound or composition than the level of the same physiological process or cellular marker in the (control) cell that has not been contacted with the composition or compound to be assayed. Analogously, a compound or composition is selected as inducing apoptosis in an antigen-presenting cell if the level of one or more physiological processes or cellular markers that decreases upon cellular apoptosis (e.g., expression of the putative apoptosis suppressive protein BCL-2) is at least about 30% lower, more preferably at least about 50%, 55%, 60%, 65%, 70%, 75%, 80%, 83%, 85%, 90%, 93%, 95%, 97%, 98%, 99% or 100%, lower, in the cell that has been contacted with the compound or composition than the level of the same physiological process or cellular marker in the (control) cell that has not been contacted with the composition or compound to be assayed.

For detailed discussion of markers of activation in antigen-presenting cells, see WO 97/14426; WO 97/24447; WO 97/29182; WO 97/29183; Paglia, P., et al., J. Exp. Med. 178:1893-1901 (1993); Paglia, P., et al., J. Exp. Med. 183:317-322 (1996); Steinman, R. M., Exp. Hematol. 24:859-862 (1996); Nestle, F. O., et al., Nature Med. 4:328-332 (1998); Geissmann, F., et al., J. Exp. Med. 187:961-966 (1998); Geissmann, F., J. Immunol. 162:4567-4575 (1999), the disclosures of all of which are incorporated herein by reference. For detailed discussion of markers of apoptosis in antigen-presenting cells, see Nagy, L., et al., Mol. Cell. Biol. 15(7):3540-3551 (1995); Horn, V., et al., FASEB J. 10: 1071-1077 (1996); Telford, W. G., et al., J. Immunol. Meth. 172(1):1-16 (1994); Campana, D., et al., Cytometry 18(2):68-74 (1994); Sgonc, R., and Wick, G., Int. Arch. Allergy Immunol. 105(4):327-332 (1994); Fraker, P. J., et al., Meth. Cell Biol. 46:57-76 (1995); Sherwood, S. W., and Schimke, R. T., Meth. Cell Biol. 46:77-97 (1995); Carbonari, M., et al., Cytometry 22(3):161-167 (1995); Mastrangelo, A. J., and Betenbaugh, M. J., Curr. Opin. Biotechnol. 6(2):198-202 (1995), the disclosures of which are incorporated by reference herein.

Thus, the invention provides high-throughput screening assays for rapidly identifying compounds or compositions that are capable of differentially modulating immune function in animal cells, particularly those compounds or compositions that are capable of activating or affecting (i.e. inhibiting/delaying/preventing, or inducing) apoptosis in antigen-presenting cells.

### Selection and Synthesis of Candidate Compounds and Compositions

As noted herein, the methods of the invention may be used to select or identify one or more compounds or compositions that are capable of modulating the immune system, preferably by affecting antigen-presenting cells. Compounds or compositions selected according to these methods can be, but are not limited to, peptides, carbohydrates, steroids and vitamin derivatives, which may each be natural or synthetic, and are preferably retinoids or retinoid derivatives that bind to one or more RARs or RXRs. The agents can be selected and screened at random, or can be rationally selected or rationally designed using protein modeling techniques. For random screening, agents such as peptides, carbohydrates, steroids or vitamin derivatives (e.g., derivatives of RA) are selected at random and are assayed, using direct or indirect methods that are routine in the art, for their ability to bind to a RAR or RXR receptor or a functional retinoid RAR:RXR receptor heterodimer. For example, candidate RAR agonists according to the present invention include synthetic retinoids such as Am80 and others for which the structures are disclosed in Ostrowski et al., Proc. Natl. Acad. Sci. USA 92:1812-1816 (1995), Roy et al., Mol. Cell. Biol. 15(12):6481-6487 (1995), and Taneja, R., et al., Proc. Natl. Acad. Sci. USA 93:6197-6202 (1996), the disclosures of which are incorporated herein in their entireties. Candidate RXR agonists according to the present invention include synthetic retinoids such as SR11237 (the structure of which is disclosed in Lehman, J. M., et al., Science 258:1944-1946 (1992), which is incorporated herein in its entirety). Other candidate RAR agonists and antagonists, and RXR agonists and antagonists, which may be used in conjunction with the methods of the invention are described in U.S. Pat. No. 5,559,248, and in commonly owned, co-pending U.S. application Ser. Nos. 08/919,318, filed Aug. 28, 1997, and 09/065,904, filed Apr. 24, 1998, and the corresponding PCT publications WO 98/08546 and WO 98/48055, the disclosures of all of which are incorporated by reference herein in their entireties.

Alternatively, agents may be rationally selected. As used herein, an agent is said to be “rationally selected” when the agent is chosen based on the physical structure of a known ligand of a RAR or RXR receptor or a functional heterodimeric RAR:RXR retinoid receptor. For example, assaying compounds possessing a retinoic acid-like structure would be considered a rational selection since retinoic acid-like compounds are known to bind to a variety of retinoid receptor heterodimers.

Since highly purified RAR and RXR proteins are now available, X-ray crystallography and NMR-imaging techniques, or techniques based on a computer model of the LBD of one or more RAR or RXR receptor types, can be used to identify the structure of the ligand binding site present on these proteins and, by extension, that which is specifically present on one or more RAR or RXR receptor types. For example, the crystal structure of the ligand binding domains of certain nuclear receptors have been described. In particular, the crystal structure of the RXR LBD is described in Bourguet et al., Nature 375:377-382 (1995), and the crystal structure of the RAR LBD is described in Renaud et al., Nature 378:681-689 (1995). Using information from the crystal structure of a RAR or RXR, computer programs are available that allow one to “rationally design” candidate agonists which would likely bind to the receptorligandbinding domains (Hodgson, Biotechnology 8:1245-1247 (1990); Hodgson, Biotechnology 9:609-613 (1991)). Suitable computer program packages for this purpose include WHAT IF (Vriend, G., J. Mol. Graphics 8:52-56 (1990)), and GRID (Goodford, J. Med. Chem. 28:849-857 (1985)). Using the predicted structure obtained via such computer modeling, candidate agonist compounds may be generated by methods of synthetic organic and inorganic chemistry that are known in the art.

Other retinoids, such as RAR and RXR agonists (which may be synthetic retinoids), suitable for use in the present invention may be prepared by the methods described and cited herein, and by others that will be familiar and routine to those of ordinary skill in the art.

### Cytokines

In certain embodiments as described herein, the methods and compositions of the invention may optionally include the use of one or more cytokines in conjunction with one or more retinoids. Preferred cytokines for use in such aspects of the invention include any compound that acts as a biological response modifier and induces a physiological response in a cell, such as growth, differentiation, senescence, apoptosis, cytotoxicity or antibody secretion, including but not limited to growth factors (such as EGF, ECGF (also known as aFGF), bFGF, KGF, HGF, IGF-1, IGF-2, TGF-β, NGF and the like), interleukins (including IL-1α, IL-1β, IL-2, IL-3, IL-4, IL-5, IL-6, IL-7, IL-8, IL-9, IL-10, IL-11, IL-12, IL-13, IL-14, IL-15, IL-16, IL-17, IL-18, etc.), colony-stimulating factors (including GM-CSF, M-CSF, G-CSF and the like), interferons (including IFN-α, IFN-β, and IFN-γ), tumor necrosis factors (particularly TNF-α), lymphotoxin, and the like. Particularly preferred for use in accordance with the present invention are IL-1β and TNFα, and functionally active fragments, variants and derivatives thereof, and most particularly TNFα.

Cytokines used in accordance with the invention preferably are isolated, and may be obtained from natural (i.e., cellular) sources such as activated monocytes, macrophages, lymphocytes or other cells. Alternatively, since the nucleotide sequences for cytokines are known in the art (e.g., the DNA sequences for the IL-1β and TNFα genes are available as GenBank Accession Nos. X04500 and E02870, respectively, which are incorporated by reference herein), cytokines may be produced using recombinant DNA techniques according to standard methods of cloning and protein expression that will be familiar to the ordinarily skilled artisan. Recombinantly produced cytokines may then be isolated using standard techniques of protein isolation and purification that are also routine in the art, particularly chromatographic and electrophoretic techniques. Such recombinant and isolated natural cytokines may also be obtained commercially, for example from Sigma (St. Louis, Mo.), R&D Systems, Inc. (Rochester, Minn.), or Life Technologies, Inc. (Rockville, Md.). As another alternative, since the three-dimensional protein structures for such cytokines are known or can be determined using routine methods of protein chemistry (e.g., X-ray crystallography, NMR, amino acid analysis, etc.), cytokines for use in the present methods and compositions may be made synthetically according to standard methods of protein synthesis, either manually or preferably using automated protein synthesis.

### Clinical Indications

Thus, methods for identifying compositions or compounds capable of differentially modulating immune system function, particularly via modulating the activation and/or apoptosis of antigen-presenting cells, are provided by the present invention. The compounds or compositions identified according to these methods may then be used, in another preferred embodiment of the invention, in methods for the treatment and/or prevention of a variety of physical disorders in animals (particularly mammals including humans) that are predisposed or susceptible to, or suffering from, a physical disorder that may be delayed, prevented, cured or otherwise treated by differentially modulating immune system function, particularly via modulating the activation and/or apoptosis of antigen-presenting cells and most particularly by modulating the activation and/or apoptosis of dendritic cells, macrophages, and/or Langerhans cells. The compositions identified by the methods of the invention may thus be used prophylactically as chemopreventive agents for such disorders. Alternatively, the compounds and compositions of the invention may be used in formulating preventative vaccines or adjuvants therefor, which may be administered to an animal to prevent the development of, or induce immunity to, a particular disease, physical disorder or disease syndrome. Certain preferred vaccines according to such aspects of the invention may comprise, for example, one or more retinoids, one or more cytokines, and one or more antigens to which an immune response is to be induced in the animal (such as one or more bacterial antigens, one or more fungal antigens, one or more viral antigens, one or more animal antigens (including one or more parasite antigens), one or more tumor cell antigens (which may be one or more tumor cell-specific antigens), one or more plant antigens, and the like, or any combinations or admixtures thereof).

According to the invention, an animal, particularly a mammal (preferably a human) that is suffering from, or that is predisposed or susceptible to, a physical disorder may be treated by administering to the animal an effective dose of a composition or compound or the invention, or a composition or compound selected according to the above-described methods of the invention optionally in combination with a pharmaceutically acceptable carrier or excipient therefor. As used herein, an animal that is “suffering from” a particular physical disorder is defined as an animal that exhibits one or more overt physical symptoms of the disorder that are typically used in the diagnosis or identification of the disorder according to established medical and veterinary procedures and protocols that will be familiar to the ordinarily skilled artisan. Analogously, as used herein, an animal that is “predisposed to” or “susceptible to” a physical disorder is defined as an animal that does not exhibit a plurality of overt physical symptoms of the disorder but that is genetically, physiologically or otherwise at risk for developing the disorder under appropriate physiological and environmental conditions. Hence, whether or not a particular animal is “suffering from,” “predisposed to” or “susceptible to” a particular physical disorder will be apparent to the ordinarily skilled artisan upon determination of the medical history of the animal using methods that are routine in the medical and veterinary arts.

Physical disorders treatable or preventable with the compositions and methods of the present invention include any physical disorder that may be delayed, prevented, cured or otherwise treated by modulating immune system function, particularly activation and/or apoptosis in antigen-presenting cells, in an animal suffering from, or predisposed or susceptible to, the physical disorder. Such physical disorders that may be treatable or preventable using compositions and methods that activate, or inhibit/delay/prevent apoptosis in, antigen-presenting cells may include, but are not limited to, infectious diseases (particularly bacterial diseases (including without limitation meningitis, pneumonia, tetanus, cholera, typhoid fever, staphylococcal skin infections, streptococcal pharyngitis, scarlet fever, pertussis, diphtheria, tuberculosis, leprosy, rickettsial diseases, bacteremia, bacterial venereal diseases and the like), viral diseases (including without limitation meningitis, AIDS, influenza, rhinitis, hepatitis, polio, pneumonia, yellow fever, Lassa fever, Ebola fever and the like), and/or fungal diseases (including without limitation cryptococcosis, blastomycosis, mucormycosis, histoplasmosis, aspergillosis, and the like), parasitic diseases (including without limitation malaria, Leishmaniasis, filariasis, trypanasomiasis, schistosomiasis, and the like), cancers (such as carcinomas, melanomas, sarcomas, leukemias and the like), and other disorders treatable or preventable using the methods and compositions of the invention that activate and/or inhibit/delay/prevent apoptosis in antigen-presenting cells in the animal. Analogously, physical disorders that may be treatable or preventable using compositions and methods that induce apoptosis in antigen-presenting cells may include, but are not limited to, immune system disorders (such as rheumatoid arthritis, multiple sclerosis, systemic lupus erythematosis, Crohn's Disease), and other disorders treatable or preventable using the methods and compositions of the invention that induce apoptosis in antigen-presenting cells in the animal. The methods of the present invention may also be used in the prevention of disease progression, such as in chemoprevention of the progression of a premalignant lesion to a malignant lesion, and to treat an animal suffering from, or predisposed to, other physical disorders that respond to treatment with compositions that activate, or inhibit/delay/prevent or induce apoptosis in, antigen-presenting cells.

### Formulation and Methods of Administration

As indicated above, the compositions and compounds of the invention, or compositions and compounds selected according to the methods of the invention, are known to elicit a wide array of cellular responses, several of which have clinical applications in treating or preventing physical disorders in a patient. The term “patient” as used herein is defined as an animal, preferably a mammal, including a human. As used herein, “an effective amount” of a retinoid or of a cytokine is defined as an amount effective to elicit a cellular response in cells which express a retinoid (and, in certain embodiments, a cytokine) receptor. Example clinical therapies which involve administering at least one retinoid and optionally at least one cytokine, or compositions comprising at least one retinoid and optionally at least one cytokine, to a patient are discussed in more detail below.

The therapeutic methods of the invention thus use one or more compounds or pharmaceutical compositions comprising one or more compounds selected for their ability to modulate immune system function, particularly via activating, or inhibiting/delaying/preventing or inducing apoptosis in, antigen-presenting cells as described herein, and optionally a pharmaceutically acceptable carrier or excipient therefor. The compositions and compounds of the invention may be administered orally, rectally, parenterally, intrasystemically, intravaginally, intraperitoneally, topically (as by powders, ointments, drops or transdermal patch), bucally, or as an oral or nasal spray. The compositions of the invention comprising one or more cytokines, or the one or more cytokines used in the methods of the invention, are preferably administered to the animal intramuscularly, subcutaneously, or intravenously, although other modes and methods of administration described herein and known to the ordinarily skilled artisan may also suitably be used in accordance with the invention. By “pharmaceutically acceptable carrier, diluent or excipient” is meant a non-toxic solid, semisolid or liquid filler, diluent, encapsulating material or formulation auxiliary of any type. The term “parenteral” as used herein refers to modes of administration which include intravenous, intramuscular, intraperitoneal, intrasternal, subcutaneous and intraarticular injection and infusion.

Pharmaceutical compositions used in the methods of the present invention for parenteral injection can comprise pharmaceutically acceptable sterile aqueous or nonaqueous solutions, dispersions, suspensions or emulsions as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol, and the like), carboxymethylcellulose and suitable mixtures thereof, vegetable oils (such as olive oil), and injectable organic esters such as ethyl oleate. Proper fluidity can be maintained, for example, by the use of coating materials such as lecithin, by the maintenance of the required particle size in the case of dispersions, and by the use of surfactant.

The pharmaceutical compositions used in the methods of the present invention may also contain adjuvants such as preservatives, wetting agents, emulsifying agents, and dispersing agents. Prevention of the action of microorganisms may be ensured by the inclusion of various antibacterial and antifungal agents, for example, paraben, chlorobutanol, phenol sorbic acid, and the like. It may also be desirable to include isotonic agents such as sugars, sodium chloride, and the like. Prolonged absorption of the injectable pharmaceutical form may be brought about by the inclusion of agents which delay absorption such as aluminum monostearate and gelatin.

In some cases, in order to prolong the effect of the drugs, it is desirable to slow the absorption from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Injectable depot forms are made by forming microencapsule matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides). Depot injectable formulations are also prepared by entrapping the drug in liposomes or microemulsions which are compatible with body tissues.

The injectable formulations can be sterilized, for example, by filtration through a bacterial-retaining filter, or by incorporating sterilizing agents in the form of sterile solid compositions which can be dissolved or dispersed in sterile water or other sterile injectable medium just prior to use.

Solid dosage forms for oral administration include capsules, tablets, pills, powders, and granules. In such solid dosage forms, the active compounds are mixed with at least one item pharmaceutically acceptable excipient or carrier such as sodium citrate or dicalcium phosphate and/or a) fillers or extenders such as starches, lactose, sucrose, glucose, mannitol, and silicic acid, b) binders such as, for example, carboxymethylcellulose, alginates, gelatin, polyvinylpyrrolidone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and I) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hardfilled gelatin capsules using such excipients as lactose or milk sugar as well as high molecular weight polyethylene glycols and the like.

The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract, optionally, in a delayed manner. Examples of embedding compositions which can be used include polymeric substances and waxes.

The active compounds can also be in micro-encapsulated form, if appropriate, with one or more of the above-mentioned excipients.

Liquid dosage forms for oral administration include pharmaceutically acceptable emulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, and perfuming agents.

Suspensions, in addition to the active compounds, may contain suspending agents as, for example, ethoxylated isostearyl alcohols, polyoxyethylene sorbitol and sorbitan esters, microcrystalline cellulose, aluminum metahydroxide, bentonite, agar-agar, and tragacanth, and mixtures thereof.

Topical administration includes administration to the skin or mucosa, including surfaces of the lung and eye. Compositions for topical administration, including those for inhalation, may be prepared as a dry powder which may be pressurized or non-pressurized. In nonpressurized powder compositions, the active ingredients in finely divided form may be used in admixture with a larger-sized pharmaceutically acceptable inert carrier comprising particles having a size, for example, of up to 100 μm in diameter. Suitable inert carriers include sugars such as lactose. Desirably, at least 95% by weight of the particles of the active ingredient have an effective particle size in the range of 0.01 to 10 μm.

Alternatively, the pharmaceutical composition may be pressurized and contain a compressed gas, such as nitrogen or a liquefied gas propellant. The liquefied propellant medium and indeed the total composition is preferably such that the active ingredients do not dissolve therein to any substantial extent. The pressurized composition may also contain a surface active agent. The surface active agent may be a liquid or solid non-ionic surface active agent or may be a solid anionic surface active agent. It is preferred to use the solid anionic surface active agent in the form of a sodium salt.

A further form of topical administration is to the eye. In this form of administration, the compounds or compositions of the invention are delivered in a pharmaceutically acceptable ophthalmic vehicle, such that the compounds or compositions are maintained in contact with the ocular surface for a sufficient time period to allow the compounds to penetrate the corneal and internal regions of the eye, as for example the anterior chamber, posterior chamber, vitreous body, aqueous humor, vitreous humor, cornea, iris/ciliary, lens, choroid/retina and sclera. The pharmaceutically acceptable ophthalmic vehicle may, for example, be an ointment, vegetable oil or an encapsulating material.

Compositions for rectal or vaginal administration are preferably suppositories which can be prepared by mixing the compounds or compositions of the invention with suitable non-irritating excipients, diluents or carriers such as cocoa butter, polyethylene glycol or a suppository wax which are solid at room temperature but liquid at body temperature and therefore melt in the rectum or vaginal cavity and release the drugs.

The pharmaceutical compositions used in the present therapeutic methods may also be administered in the form of liposomes. As is known in the art, liposomes are generally derived from phospholipids or other lipid substances. Liposomes are formed by mono- or multi-lamellar hydrated liquid crystals that are dispersed in an aqueous medium. Any non-toxic, physiologically acceptable and metabolizable lipid capable of forming liposomes can be used. The present pharmaceutical compositions in liposome form can contain, in addition to the one or more retinoids and one or more cytokines, stabilizers, preservatives, excipients, and the like. The preferred lipids are the phospholipids and the phosphatidyl cholines (lecithins), both natural and synthetic. Methods to form liposomes are known in the art (see, e.g., Prescott, E., Meth. Cell Biol. 14:33 (1976)).

### Dosaging

By the invention, one or more retinoids and one or more cytokines can be administered in vitro, ex vivo or in vivo to antigen-presenting cells to enhance the immune response in an animal by activating the antigen-presenting cells. One of ordinary skill will appreciate that effective amounts of a retinoid and/or cytokine can be determined empirically and may be employed in pure form or, where such forms exist, in pharmaceutically acceptable salt, ester or prodrug form. The retinoid(s) and cytokine(s) may be administered to a patient in need thereof as pharmaceutical compositions in combination with one or more pharmaceutically acceptable excipients. It will be understood that, when administered to a human patient, the total daily usage of the compounds and compositions of the present invention will be decided by the attending physician within the scope of sound medical judgement. The specific therapeutically effective dose level for any particular patient will depend upon a variety of factors including the type and degree of the cellular response to be achieved; activity of the specific retinoid(s) and cytokine(s) employed; the specific composition employed; the age, body weight, general health, sex and diet of the patient; the time of administration, route of administration, and rate of excretion of the retinoid(s) and cytokine(s); the duration of the treatment; drugs used in combination or coincidental with the specific retinoid(s) and cytokine(s); and like factors well known in the medical arts. For example, it is well within the skill of the art to start doses of retinoid(s) and cytokine(s) at levels lower than those required to achieve the desired therapeutic effect and to gradually increase the dosages until the desired effect is achieved.

For example, satisfactory results are obtained by oral administration of a retinoid at dosages on the order of from 0.05 to 10 mg/kg/day, preferably 0.1 to 7.5 mg/kg/day, more preferably 0.1 to 2 mg/kg/day, administered once or, in divided doses, 2 to 4 times per day. On administration parenterally, for example by i.v. drip or infusion, dosages on the order of from 0.01 to 5 mg/kg/day, preferably 0.05 to 1.0 mg/kg/day and more preferably 0.1 to 1.0 mg/kg/day can be used. Suitable daily dosages for patients are thus on the order of from 2.5 to 500 mg p.o., preferably 5 to 250 mg p.o., more preferably 5 to 100 mg p.o., or on the order of from 0.5 to 250 mg i.v., preferably 2.5 to 125 mg i.v. and more preferably 2.5 to 50 mg i.v. Suitable daily dosage ranges for the one or more cytokines used in the compositions or methods of the invention (which may advantageously be administered intramuscularly, subcutaneously or intravenously, or by any other suitable mode or method of administration familiar to those skilled in the art) are on the order of about 0.1 unit to about 100,000,000 units, about 0.5 unit to about 50,000,000 units, about 1 unit to about 25,000,000 units, about 5 units to about 20,000,000 units, about 10 units to about 15,000,000 units, about 15 units to about 10,000,000 units, about 20 units to about 750,000 units, about 25 units to about 500,000 units, about 50 units to about 250,000 units, about 50 units to about 100,000 units, about 50 units to about 50,000 units, about 50 units to about 25,000 units, about 50 units to about 10,000 units, about 50 units to about 5,000 units, about 50 units to about 2,500 units, about 50 units to about 11,000 units, about 50 units to about 500 units, or about 100 units to about 500 units. In particular, TNFα is preferably administered in the compositions and methods of the invention at amounts ranging from about 10 units to about 15,000 units, about 15 units to about 10,000 units, about 20 units to about 7500 units, about 25 units to about 5000 units, about 50 units to about 2500 units, about 50 units to about 1000 units, about 50 units to about 500 units, or about 100 units to about 500 units, and most preferably at about 50 units to about 1000 units. If desired, TNFα may be administered in conjunction with one or more additional cytokines (e.g., IL-1β), at the above dosage ranges or at dosage ranges described in reports of clinical studies available in the literature (see, e.g., Eskander et al., Am. J. Clin. Onol. 20:511-514 (1997), which is incorporated herein by reference for its relevant teachings). Of course, other suitable dosages for use in accordance with the invention may be determined on a patient-by-patient basis by those of ordinary skill in the pharmaceutical arts, using assays that are well-known in the art without resorting to undue experimentation (see, e.g., Eskander et al., Am. J. Clin. Onol. 20:511-514 (1997); Harris et al., Sem. Oncol. 26:439-447 (1999), which are incorporated herein by reference for their relevant teachings). Assays for determining activity (units/mg) of a given cytokine preparation will be familiar to those of ordinary skill in the art. For instance, the activity of a preparation of TNFα may be determined using a standard cytotoxicity assay using L929 or WEHI target cells, as described, for example, in Gautam et al., J. Hematotherapy 8:237-245 (1999) and in Postma et al., Antimicrob. Agents Chemother. 43:1027-1033 (1999), which are incorporated herein by reference for their relevant teachings. Other suitable assays for determining the activity of preparations of other cytokines that may be advantageously used in the compositions and methods of the invention will be familiar to one of ordinary skill.

Dosaging of the retinoids and/or cytokines may also be arranged in a patient-specific manner to provide a predetermined concentration of a retinoid and/or cytokine in the blood, as determined by techniques accepted and routine in the art (HPLC is preferred). Thus patient dosaging may be adjusted to achieve regular on-going retinoid blood levels, as measured by HPLC, on the order of from 50 to 1000 ng/ml, preferably 150 to 500 ng/ml.

It will be readily apparent to one of ordinary skill in the relevant arts that other suitable modifications and adaptations to the methods and applications described herein are obvious and may be made without departing from the scope of the invention or any embodiment thereof. Having now described the present invention in detail, the same will be more clearly understood by reference to the following examples, which are included herewith for purposes of illustration only and are not intended to be limiting of the invention.

### EXAMPLES

### Materials and Methods

The following materials and methods were generally used in all Examples:

Culture of LC. Pure (≧95%) immature dendritic cells with the Langerhans cell phenotype (CDla⁺, E-cadherin⁺, CLA⁺, Lag⁺, CMHII^{lo}, CD86⁻,TNFRI^{lo}), high pinocytic and weak antigen presentation activities, were obtained as previously described (Geissmann, F., et al., J. Exp. Med. 187:961-966 (1998);

Geissmann, F., J. Immunol. 162:4567-4575 (1999)) from purified blood CD14⁺ human monocytes cultured for 5 to 7 days in RPMI 1640 supplemented with 2 mM L-glutamine, 100 U/ml penicillin, 100 mg/ml streptomycin, 10% heat-inactivated FCS Myoclone (all from Life Technologies, Inc., Rockville, Md.), 250 ng/ml GM-CSF (Sandoz AG Bale, Switzerland), 30 ng/ml IL-4 and 10 ng /ml TGFβ1 (R&D Systems, Minneapolis, Minn.), and are referred to herein as LC. These immature DC become mature after CD40L triggering (as a surrogate cognate signal) (Geissmann, F., J Immunol. 162:4567-4575 (1999)).

Retinoids and cytokines. All-trans-RA (tRA) was from Sigma Immunochemicals (St. Louis, Mo.), and retinol and 9-cis-RA (9cRA) were from Calbiochem, La Jolla, Calif. Agonists for RARα (Compound II), RARβ (Compound III and Compound VIII), RARγ (Compound IV), RARα (Compound II) and pan-RAR (Compound VIII) antagonists, and a pan-RXR agonist (SR11237) were a gift from Bristol-Myers Squibb. All retinoids were stored dissolved in ethanol at 10⁻³M. Human recombinant TNFα and IL-1β were from R&D Systems, Minneapolis, Minn. Murine fibroblast cell lines transfected with human CD40L was kindly provided by Dr F. Brière (Schering-Plough, Dardilly, France).

Transcriptional Activity Assays. Transcriptional activities of retinoids used in these studies (results depicted in Table 3) have been previously determined using reporter cells for the AF2 functions of RARs (α, β or γ), and RXRs, while RXRα homodimer [(RXR)₂] activity on a DR1-tk-CAT reporter gene and RARα-RXRα heterodimer activity on a DR5-tk-CAT reporter gene were determined by transient transfections (Chen, J. -Y., et al., Nature 382:819-822 (1996); Chazaud, C., et al., Development 126:2589-2596 (1999); Taneja, R., et al., Proc. Natl. Acad. Sci. USA 93:6197-6202 (1996); Chen, J. -Y., et al., EMBO J. 14:1187-1197 (1995)).

Flow Cytometry Analysis. 1×10⁵ cells were incubated in 96-well plates (Becton Dickinson Labware, Franklin Lakes, N.J.) for 15 min at 4 ° C. in PBS, 2% human AB serum, and 0.01 M NaN₃, with FITC-conjugated anti-DR (Immunotech, Marseille, France) and PE-conjugated CD86 (Pharmingen, San Diego, Calif.) at the appropriate concentration, or with control isotype-matched irrelevant mAbs at the same concentration. After washing, 10⁴ events were analyzed with a FACScalibur (Becton Dickinson Immunocytometry Systems, Mountain View, Calif.) using CellQuest software.

Antigen presentation assays. Dendritic cells were collected, washed three times, pulsed for 8 hours with tetanus toxin or medium alone, then for 40 hours with or without retinoids and TNFα (10 ng/ml). Cells were washed 2 times in RPMI with 10% human AB serum, resuspended in RPMI with 10% human AB serum and added in triplicate at various concentrations to 10⁵ autologous (autologous antigen presentation assay) or allogenous (MLR) T-cells/well in 96-well tissue culture plates (Falcon). T-cells were isolated by the standard Ficoll-Paque method followed by magnetic depletion of non-T cells (MACS; Miltenyi Biotec, Berglsch Gladbach, Germany). [³H]Thymidine (Amersham Life Science, Buckinghamshire, UK) incorporation was measured in newly synthesized DNA over 18 h, using pulses initiated at day 4 or 5 of the culture with 1 mCi/well of [³H]thymidine. Cells were then harvested with a 96-well Harvester (Pharmacia, St. Quentin, France), collected on glass-fiber filter (Pharmacia) and the incorporation of thymidine was measured with a Beta-plate microscintillation counter (LKB, Pharmacia).

Quantitation of IL-12 production by ELISA. Supernatants were stored at −70° C. until cytokine measurements. Production of bioactive IL-12 p70 was measured in duplicate using ELISA Quantikine Kits (R&D Systems, Minneapolis, Minn.) according to the manufacturer's instructions. Sensitivity of IL-12 detection was 0.5 pg/ml.

Electrophoretic Mobility Shift Assay. Cells (4×10⁶) were washed once in cold PBS, and allowed to swell on ice for 10 min in buffer A (10 mM HEPES, pH 7.9, 1.5 mM MgCl₂, 10 mM KCl, 0.5 mM DTT and 2 mM PMSF) containing the following protease inhibitors: leupeptin, aprotinin, pepstatin and antipain, each at a concentration of 4 μg/ml. Samples were then centrifuged and the pellet suspended in 20 μl of buffer C. (20 mM HEPES, pH 7.8,420 mM NaCl, 1.5 mM MgCl₂, 0.5 mM DTT and 2 mM PMSF) containing the protease inhibitors. The mixture was left for 20 min on ice. Cell debris was removed by centrifugation and the resulting nuclear extracts were stored at −70° C. Protein concentration was measured with the BioRad protein assay (BioRad Laboratories, Munich, Germany). Nuclear extracts (5 μg to 10 μg protein) were assayed for DNA-binding activity in a total volume of 20 μl of binding buffer (20 mM Tris-HCl, pH 8, 60 mM KCl, 2 mM MgCl₂, 0.3 mM DTT, 12% glycerol, and 3 μg poly dI-dC). Nuclear extracts were incubated separately for 15 minutes at 30° C. with the double-stranded labeled probes for NF-κB (5′-GATCCCAAGAGGGATTTCACCTAAATCC-3′) (SEQ ID NO:1). The samples were then loaded onto a non-denaturing 5% polyacrylamide gel and subjected to electrophoresis at 14 V/cm in a low-ionic-strength buffer (0.5×TBE). Gels were dried and examined with a Phosphorimager (Molecular Dynamics, Sunnyvale, Calif.).

Statistics. Student t test was used to interpret the significance of differences between experimental groups (presented as mean±s.d.) P value was two tailed and was considered significant when <0.05.

### Example 1

### Physiological Levels of Vitamin A Result in Increased Apoptosis of Immature Dendritic Cells, an Effect Mediated Through Nuclear Retinoic Acid Receptors

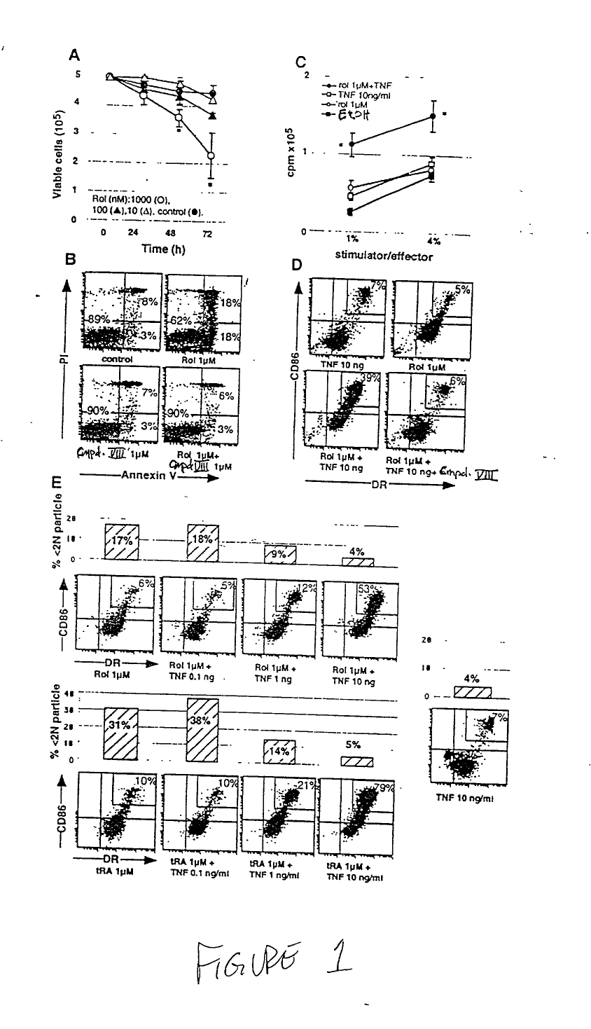

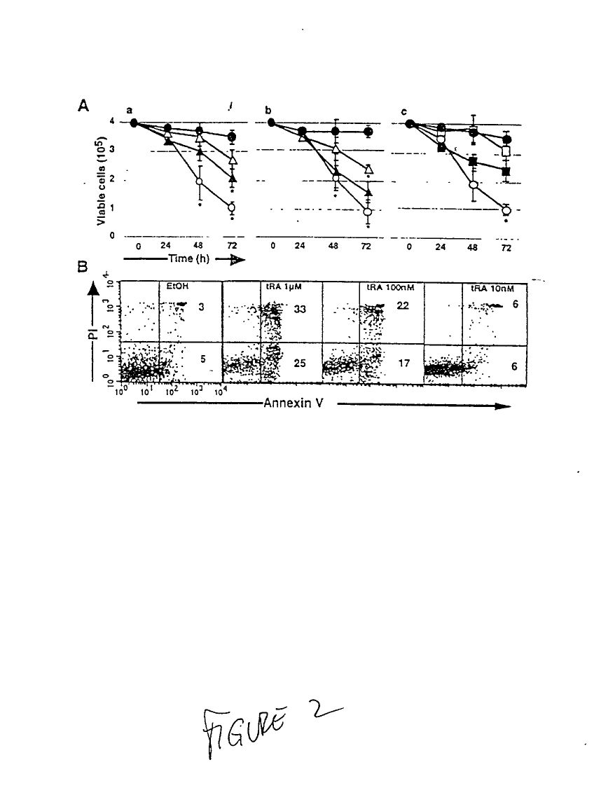

To determine the effects of vitamin A on survival of immature dendritic cells (DC), Langerhans cells (LC) were produced as described above in the Materials and Methods, treated with varying concentrations of retinol (Rol), and cells were tested for viability and other markers of apoptosis. 1 μM Rol significantly increased immature LC death, with a 20% and 40% reduction of viable cell numbers after two and three days, respectively (FIG. 1A). At day two, 17±8% of the cells were apoptotic, vs. <5% in controls (p<0.01), as determined by flow cytometry analysis of PI stained nuclei and annexin V/PI staining of cells (FIG. 1B; Table 3A). The active derivatives of retinol, all-trans retinoic acid (tRA) and 9-cis retinoic acid (9cRA), induced a similar dose- and time-dependent increase in death, albeit at lower (100 nM) concentration (FIGS. 2A, B and Table 2). A pan-RAR (α, β and γ) antagonist (Compound VIII (see WO 98/46228, the disclosure of which is incorporated herein by reference; see also Chazaud, C., and Chambon, P., Development 126:2589-2596 (1999), and Tables 1 and 2 herein) inhibited retinol-induced cell death (FIG. 1B, Table 3C), indicating that the effect of retinol was mediated through RAs and their receptors. LC death was also blocked by the caspase inhibitor Z-Vad-fmk (FIG. 2A and Table 3C), but not by anti-Fas blocking antibody ZB4. Since LC express Fas, these results indicate that a FasL-independent mechanism is involved in retinol-induced apoptosis of LC. This effect was restricted to DC at their immature stage of differentiation, because neither mature DC (CD40L-treated) nor monocytes (from day 0 to day 4 of culture with cytokines) died after exposure to retinoids.

TABLE 2
Functional characteristics of retinoids.

| | RARα | RARβ | RARγ | RXR | (RXR)₂ | RARα-RXR |
|---|---|---|---|---|---|---|
| Ligands | AF2 | AF2 | AF2 | AF2 | AF2 | DR5 |
| Natural ligands | | | | | | |
| tRA | +^{(a)} | + | + | 0^{(a)} | 0 | + |
| 9cRA | + | + | + | + | + | + |
| Synthetic ligands | | | | | | |
| Compound I | + | 0 | 0 | 0 | 0 | + |
| Compound II | −^{(a)(b)} | 0 | 0 | 0 | 0 | − |
| Compound III | 0 | + | 0 | 0 | nd^{(a)} | nd |
| Compound VII | − | + | − | 0 | nd | nd |
| Compound IV | 0 | (+)^{(a)} | + | 0 | 0 | 0 |
| Compound VIII | − | − | − | 0 | nd | nd |
| SR11237 | 0 | 0 | 0 | + | + | 0 |
| Compound V | − | − | − | + | + | − |

^{a)}Retinoid activity: “+” = agonist, “(+)” = weak agonist, “−” = antagonist; “0” = absence of agonist and antagonist activities; “nd” = not determined.
^{b)}In some instances, Compound II may synergize with a RXR agonist (Chen, JY., et al., Nature 382:819-822 (1996)).

Table 3. Retinol-induced apoptosis is mediated via RARα-RXR heterodimers and is inhibited by caspase inhibitors and TNFα.

TABLE 3A
Retinol, retinoic acids and a selective RARα ligand (Compound I)
induce death of immature LC.

| | Controls | | | | Compound | | | | | | |
|---|---|---|---|---|---|---|---|---|---|---|---|
| Conc. | EtOH | Rol | tRA | 9cRA | I | VII | III | IV | SR11237 | II | V |
| 1 nM | — | ≦5^{(a)} | ≦5 | ≦5 | ≦5 | ≦5 | ≦5 | ≦5 | ≦5 | ≦5 | ≦5 |
| 10 nM | — | ≦5 | ≦5 | ≦5 | ≦5 | ≦5 | ≦5 | ≦5 | ≦5 | ≦5 | ≦5 |
| 50 nM | — | — | — | — | 18 ± 3^{(b)} | — | — | — | — | — | — |
| 100 nM | — | ≦5 | 14 ± 6^{(b)} | 8 ± 3 | 27 ± 9 | ≦5 | ≦5 | ≦5 | ≦5 | ≦5 | ≦5 |
| 1 μM | ≦ | 17 ± 8 | 36 ± 10 | 30 ± 9 | 37 ± 7 | 10 ± 6 | ≦5 | ≦5 | ≦5 | ≦5 | ≦5 |

^{(a)}Values are mean ± SD percentage of apoptotic (<2N) particles after two days in culture with retinoids (day 2) determined by flow cytometry of propidium stained nuclei.
^{(b)}Italics: p <0.05; bold: p<0.01 to <0.001.

TABLE 3B
Synergy between a pan-RXR agonist (SR11237) and selective
RARα and RARβ agonists. (see Tables 1 and 2).

| | 100 nM SR11237 + Compound_{——} | | | | | |
|---|---|---|---|---|---|---|
| Conc.of Ligand | I | VII | III | II | VIII | IV |
| 1 nM | 6 ± 3 | ≦5 | ≦5 | ≦5 | ≦5 | ≦5 |
| 10 nM | 25 ± 10^{(a)} | 5 | ≦5 | ≦5 | ≦5 | ≦5 |
| 100 nM | 32 ± 7 | 20 ± 6 | 16 ± 3 | ≦5 | ≦5 | ≦5 |
| 1 μM | 47 ± 10 | 19 ± 7 | 30 ± 2 | ≦5 | ≦5 | ≦5 |

^{(a)}bold: p<0.01 to <0.001.

TABLE 3C
Inhibition of retinoid-induced apoptosis by RAR antagonists,
caspase inhibitor, TNTα and CD40L.

| | | Rol | tRA | Cmpd. I, | Compound I, 100 nM + |
|---|---|---|---|---|---|
| | 0 | 1 μM | 1 μM | 1 μM | SR11237, 100 nM |
| medium | ≦5 | 17 ± 8 | 38 ± 10 | 38 ± 8 | 38 ± 5 |
| Cmpd. II (1 μM) | ≦5 | — | 15 ± 8^{(a)} | 9 ± 5^{(a)} | 7 ± 3 |
| Cmpd. VIII (1 μM) | ≦5 | 4 ± 1 | 10 ± 6 | 5 ± 3 | 4 ± 2 |
| Z-Vad (50 μM) | ≦5 | — | 9 ± 4 | 9 ± 7 | 11 ± 4 |
| TNFα (10 ng/ml) | ≦5 | 4.5 ± 1 | 8 ± 7 | 11 ± 1 | 14 ± 2 |
| CD40L^{(b)} | ≦5 | — | ≦5 | ≦5 | ≦5 |

^{(a)}Italics: p<0.05; bold: p <0.01 to <0.001.
^{(b)}Coculture of LC with 25% irradiated CD40L transfected L cells.

### Example 2

### RARα-RXR Heterodimers Mediate Retinoid-induced Apoptosis

A panel of synthetic retinoid ligands (Table 2) was used to investigate which receptors (among RARs and RXRs) were involved in the apoptotic effect of retinol. Day 6 immature LC were cultured for 40 h with natural and synthetic retinoids or vehicle (ethanol, EtOH), caspase-inhibitor Z-Vad-fmk (50 nM) or TNFα (10 ng/ml) as indicated. Cells were washed and nuclei were stained with PI (50 μg/ml in PBS, 0.1 NaCl, 0.1% TritonX-100 for 20 min at 37° C.). The % of sub 2N particles was analyzed on 2×10⁴ events with a FACScalibur (Becton Dickinson) using CellQuest software (Becton Dickinson). Similar results were obtained with Annexin-V/PI staining, of intact cells (see FIGS. 1 and 2B). Results are depicted in FIG. 2 and Table 3 (results shown in Table 3A-C are the mean±SD of 3 to 10 experiments on different donors).

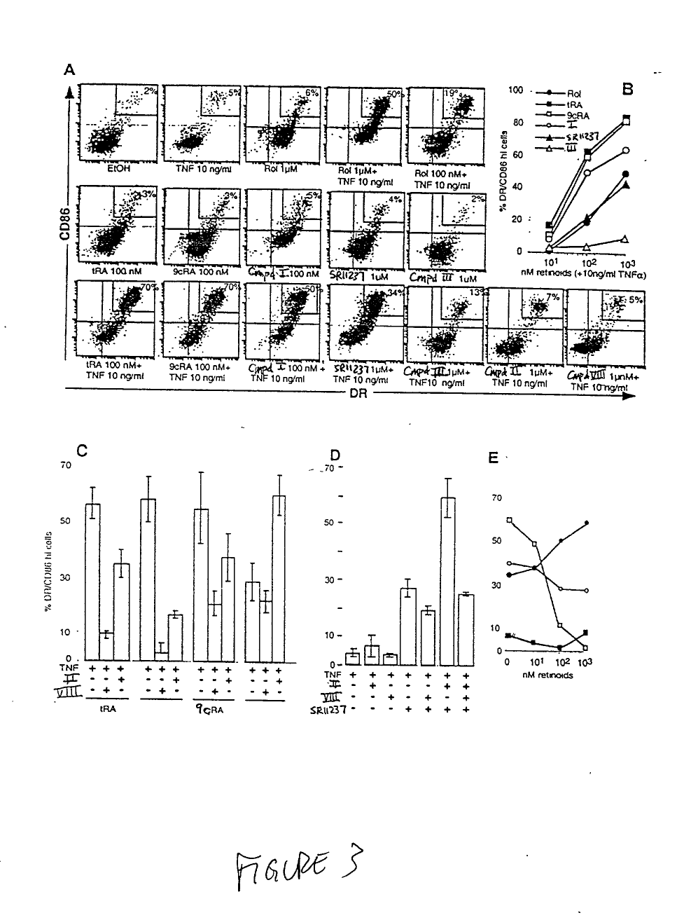

As shown in FIG. 3A and Table 3C, tRA-induced apoptosis of immature LC was inhibited by the pan-RAR antagonist Compound VIII and by a RARα-selective antagonist (Compound H; Chen, J. Y., et al., Nature 382:819-822 (1996)). Conversely, apoptosis was induced by a selective RARα agonist (Compound I; Taneja, R., et al., Proc. Natl. Acad. Sci. USA 93:6197-6202 (1996)), but not by RARβ agonists BMS 641, BMS453; Chen, J. Y., et al., EMBO J. 14:1187-1197 (1995)), a RARγ agonist (Compound IV; Taneja, R., et al., Proc. Natl. Acad. Sci. USA 93:6197-6202 (1996)), or a pan-RXR agonist (SR11237/SR11237; Chen, J. Y., et al., Nature 382:819-822 (1996); Taneja, R., et al., Proc. Natl. Acad. Sci. USA. 93:6197-6202 (1996); Lehmann, J. M., et al., Science 258:1944-1946 (1992)) (FIG. 1, Table 3A). These results indicate that the apoptotic effect of retinoic acid is transduced by RARα. Interestingly, the pan-RXR agonist (SR1 1237), which had no effects on its own, had asynergistic effect on LC death induced by the selective RARα agonist (Table 2A and B). The RARβ agonists Compound III and BMS453, but not the selective RARγ agonist (Compound IV), also synergized with the pan-RXR agonist to induce LC death (Table 3B), albeit with a lower efficiency. Thus, the retinoid signal triggering LC death is transduced by RARα/RXR heterodimers in which the transcriptional activity of RXR is subordinated to ligand binding to its RAR partner (Chen, J. Y., et al., Nature 382:819-822 (1996); Chen, J. Y., et al., EMBO J. 14:1187-1197 (1995); Vivat, V., et al., EMBO J. 16:5697-5709 (1997), Table 3)). Furthermore, RARβ-RXR heterodimers can also mediate the apoptotic effect of retinoids, but with a lower efficiency.

### Example 3

### Retinoid-induced Apoptosis is Inhibited by Inflammatory and Cognate Signals

The data shown in the preceding Examples may account, at least in part, for the in vitro inhibitory effect of retinoids on the allogenous mixed lymphocyte reaction (MLR) (Bedford, P. A. and Knight, S. C., Clin. Exp. Immunol. 75:481-486 (1989)). However, they are puzzling when considering most in vivo data available (Malkovsky, M.,et al., Nature 302:338-340 (1983); Meunier, L., et al., J. Invest. Dermatol. 103:775-779 (1994)), and appear contradictory to epidemiological data on vitamin A deficiency (Underwood, B. A. and Arthur, P., FASEB J. 10:1040-1048 (1996); Semba, R. D., Clin. Infect. Dis. 19:489-499 (1994); Rahmathullah, L., et al., N. Eng. J. Med. 323:929-935 (1990); Sommer, A., Lancet 339:864 (1992); Ross, A. C. and Stephensen, C. B., FASEB J. 10:979-985 (1996)). A major difference between in vitro and in vivo antigen presentation may be the environmental inflammatory signals encountered by DC, because pathogens or grafting usually induce local inflammation. Inflammatory cytokines, such as TNFα and IL-1β, enhance migration of immature LC from the skin to the lymph nodes (Williams, N. A. and Hill, T. J., J. Invest. Dermatol. 97:933-937 (1991); Roake, J., et al., J. Exp. Med. 181:2237-2247 (1995)) and were shown to contribute to activation of immature DC (including LC in vivo) (Banchereau, J. and Steinman, R. M., Nature 392:245-252 (1998); Sallusto, F., et al., J. Exp. Med. 182:389-400 (1995)). We therefore investigated the effect of TNFα and IL-1β in the presence of retinoids.

We found that TNFα inhibited the pro-apoptotic effects of retinol in a dose-dependent manner (>50% inhibition at 1 ng/ml of TNFα), as well as those of tRA (FIG. 1E), 9cRA and of the RARα selective agonist Compound I (Table 3C). Other stimuli known to contribute to LC activation such as IL-1β (1-10 ng/ml) and the cognate signal CD40L (Table 3C) also inhibited the apoptotic effect of retinoids. Therefore, retinoids and inflammatory or cognate signals have opposite effects on immature LC survival. In addition, the apoptotic effect of retinoids and its inhibition by the above mentioned signals was also observed on other type of immature DC (i.e., differentiated from monocytes in the presence of GM-CSF and IL-4).

### Example 4

### Synergistic Activation of Immature Dendritic Cells by Retinoids and Inflammatory Cytokines

We next investigated the effects of retinoids on immature LC activation. On its own, TNFα (as well as LPS and IL-1β) is not sufficient to induce significant activation (Class II and costimulatory molecule expression and enhancement of antigen presentation) of immature LC (Geissmann, F., et al., J. Immunol. 162:4567-4575 (1999); see also FIGS. 1E, 3A, 5), because TGF-β1 inhibits the activation of these cells in response to inflammatory stimuli (Geissmann, F., J. Immunol. 162:4567-4575 (1999); Garbe A, et al., Blood, American Society of Hematology, 40th meeting, Abstract 668 (1998)). This may account for the presence of immature LCs in the T-cell zone of skin-draining lymph nodes (Geissmann, F., J. Immunol. 162:4567-4575 (1999); Takahashi, K., et al., Am. J. Pathol. 153:745-755 (1998)), and it raises the possibility that other factors regulate immature DC activation by inflammatory cytokines.

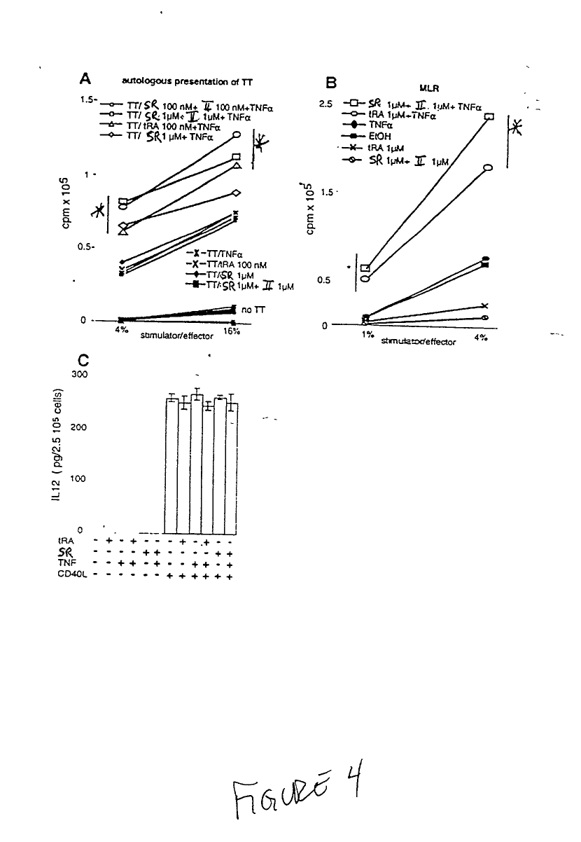

Strikingly, retinol synergized with TNFα (≧1 ng/ml) (FIGS. 1E and 3A) to induce a dose-dependent increase in the percentage of DR^{hi}-CD86^{hi} LC which parallels inhibition of apoptosis (see FIG. 1E). Rol also synergized with IL-1β (1-10 ng/ml) to activate LC, and tRA, 9cRA and the selective RARα agonist Compound I (FIGS. 1E, 3A, B) had similar effects as Rol. In addition, the selective RXR agonist SR11237 also synergized with TNFα (at 1-10 ng/ml) to activate LC (FIGS. 3A, B. In contrast, in the absence or at low concentrations of TNFα (>1 ng/ml), Rol and tRA (FIG. 1E) as well as 9cRA, RAR and RXR agonists (FIG. 3A) did not significantly induce class II or CD86 upregulation on immature LC, nor increase their antigen presentation efficiency (see below, FIGS. 1E and 4. Rather, in 6 out of 9 experiments on distinct donors, retinoids on their own further decreased the capacity of immature LC to stimulate T-cells (see FIG. 4B).

### Example 5

### Retinoids Synergize with TNFα to Enhance Antigen Presentation to T-cells

LC migrate in T-cell areas of secondary lymphoid organs where they interact with T-helper cells (Banchereau, J. and Steinman, R. M., Nature 392:245-252 (1998)). Their levels of class II and costimulatory molecule (e.g., CD86) expression influence the outcome of this interaction. To investigate the effect of retinoids on an antigen-specific immune response, we studied the presentation of tetanus toxoid (TT) by LC to fresh autologous lymphocytes of immune individuals. TT-pulsed LC exposed to retinoids and TNFα induced a four-fold increase in the proliferative response of autologous T-lymphocytes as compared with pulsed untreated LC or LC incubated with either TNFα alone or retinoids alone (FIG. 4A). LC treated with retinoids and TNFα also increased allogeneic T-lymphocyte proliferation (MLR) (FIGS. 1C and 4B).

Note that even though LC were washed several times before coculture, a direct effect of retinoids on T-cells cannot be easily ruled out because retinoids are highly lipophilic. However, such an effect appears unlikely, as tRA and/or TNFα-treated LC did not stimulate autologous T-lymphocytes in the absence of antigen (FIG. 4A). Furthermore, T-cells neither respond to externally provided retinoic acid, nor synthesize appreciable amounts of it (Buck, J., et al., Science 254:1654-1656 (1991)). We therefore conclude from the above results that retinoids synergize with inflammatory signals to induce activation of immature LC, and enhance their ability for antigen presentation.

### Example 6

### Retinoids Synergize with TNFα to Increase DNA-binding Activity of NF-κB Complexes

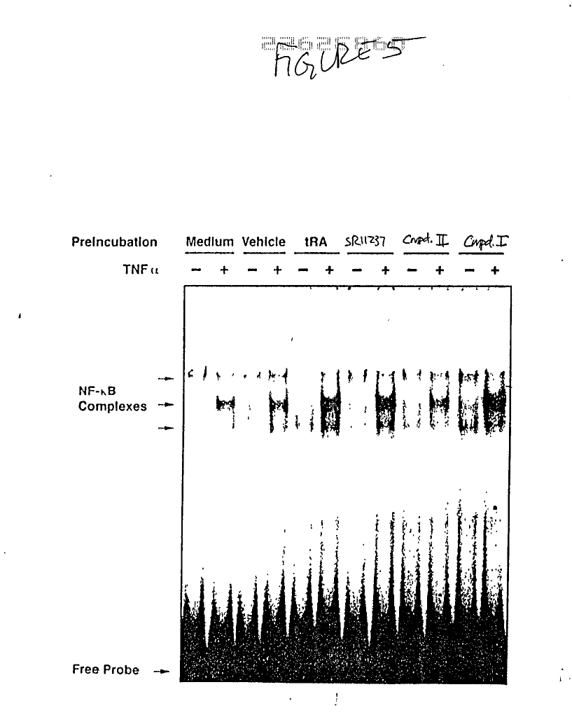

Since nuclear translocation and binding of NF-κB complexes to their DNA response element has been shown to mediate DC activation (Banchereau, J. and Steinman, R. M., Nature 392:245-252 (1998); Rescigno, M., et al., J. Exp. Med 188:2175-2180 (1998)), one possible effect of retinoids could be to increase DNA binding of NF-κB. As shown in FIG. 5, addition of TNFα after a short (6 hr) exposure to retinoids induced a strong increase in the binding of nuclear NF-κB complexes to a cognate DNA probe when compared to nuclear extracts from cells non-exposed to retinoids. Retinoids on their own had no detectable effect on NF-κB complex binding activity (FIG. 5). Interestingly, addition of TPCK (10 nM), reported by others to inhibit translocation of NF-κB and activation of DCs (Takahashi, K., et al., Am. J. Pathol. 153:745-755 (1998)), inhibited by 50% the increase of class II and CD86 on LCs treated with tRA and TNFα without any evidence of toxicity.

### Example 7

### IL-12 Production by LC is not Influenced by Exposure to Retinoids

DC activate T-cells through antigen-restricted class III/TCR and costimulatory molecule interaction (Banchereau, J. and Steinman, R. M., Nature 392:245-252 (1998)). Activated T-cells then in turn activate DC/LC through CD40L/CD40 interaction (Ridge, J., et al., Nature 393:474-478 (1998); Banchereau, J. and Steinman, R. M., Nature 392:245-252 (1998)), resulting in the production of IL-12 by DC/LC (Cella, M., et al., J. Exp. Med. 184:747-457 (1996); Banchereau, J. and Steinman, R. M., Nature 392:245-252 (1998)). We investigated whether retinoids may influence IL-12 production by LC at the cellular level. Neither TNF α]or retinoids alone, nor the combination of the two, at doses which induce apoptosis or activation, induced production of significant levels of IL-12 in culture supernatants in the absence of CD40 triggering (FIG. 4C). CD40 triggering resulted in the production of IL-12 by LC, which was not modulated by retinoids and TNFα (FIG. 5). Thus retinoids, that enhance the early phase of LC/T-cell antigen-restricted interaction mediated through class II/TCR and costimulatory molecules, may not replace, nor influence directly, the late issue of the LC/T-cell interaction mediated trough CD40LICD40 interaction.

### Example 8

### Distinct Retinoid Receptor Pathways Mediate the Effects of Retinoids on TNFα-induced LC Activation

The pan-RXR agonist (SR11237) synergized with TNFα to induce expression of CD86 and class II antigen (FIG. 3A), to increase DNA-binding activity of NF-κB complexes (FIG. 5) and to enhance presentation to antigen-specific T-cells (FIG. 4). The pan-RAR antagonist (Compound VIII) had only a weak effect on this TNFα-SR11237 synergism for LC activation (FIGS. 3C-E). In contrast, Compound VIII strongly inhibited tRA-induced and Compound I-induced apoptosis (Table 3), as well as RAR-dependent activation of LC (see below). Moreover, another synthetic retinoid (BMS749), that acts as both a pan-RXR agonist and apan-RAR antagonist (Benoit et al, manuscript submitted), also synergized with TNFα to activate LC. Note that neither SR11237 (Table 3) nor BMS749 exhibited an apoptotic effect on their own. Thus, activation of immature LC in synergy with TNFα may be achieved via a RXR pathway that is RAR-independent and not involved in apoptosis.

On the other hand, the synergistic effects between TNFα and either retinol, tRA or the RARα agonist Compound I on LC activation were inhibited by the pan-RAR antagonist Compound VIII (FIGS. 1D, 3C), demonstrating that a RARα-dependent pathway could also induce activation. Interestingly, the RARα antagonist Compound II synergized with the RXR agonist (SR11237) for TNFα-induced LC activation (FIGS. 3C-E) and antigen presentation (FIGS. 4A, B). This synergistic effect was inhibited by Compound VIII (FIG. 3D), therefore indicating the involvement of RXR/RARα heterodimers. Note that Compound II did not induce, but inhibited retinoid-induced apoptosis (Table 3A-C).

Altogether these results demonstrate that the effects of retinoids on LC activation are efficiently transduced through both (I) an RXR pathway that is RAR-independent, and (ii) an RARα/RXR heterodimer-dependent pathway, the latter responding to bona fide RARα agonists (tRA and Compound I), as well as to the RARα antagonist Compound II in the presence of the pan-RXR agonist SR11237. The RARβ agonists (Compound III and BMS453) had a weak effect on activation of immature LC (FIG. 3A) and the selective RARγ agonist (Compound IV) had no effect whatsoever. Interestingly, the pan-RAR antagonist Compound VIII only partially blocked 9cRA-induced activation (FIG. 3C), to a level similar to that obtained with the RXR agonist SR11237. These results, considered together with the fact that 9cRA binds to both RAR and RXR, suggest that 9cRA is able to trigger both the RAR-dependent and the RAR-independent pathways of LC activation.

### General Discussion

Our present data demonstrate that vitamin A (retinol) acts at physiological concentration on immature dendritic cells such as Langerhans cells, to regulate either apoptosis or antigen presentation in cooperation with inflammatory cytokines, and that these effects are mediated by its active derivatives and their cognate nuclear receptors. This identifies immature dendritic cells as a key cellular target of vitamin A in the immune system.

DC are essential antigen-presenting cells that initiate immune responses. Immature Langerhans cells internalize antigens and, after being activated by inflammatory stimuli and other indicators of cell damage, migrate to T-cell areas of secondary lymphoid organs where they foster the egress of effector T-cells in the periphery (Banchereau, J. and Steinman, R. M., Nature 392:245-252 (1998)). DC also stimulate B-cell (Banchereau, J. and Steinman, R. M., Nature 392:245-252 (1998)), NKT (Kawano, T., et al., Science 278:1626-1629 (1997)) and NK (Fernandez, N. C., et al., Nat. Med. 4:405-411 (1999)) immune response. Epithelial cells produce active TGFβ1 (Munger, J. S., et al., Cell 96:319-28 (1999)), which is required for the differentiation of Malpighian-epithelium associated immature Langerhans cell differentiation (Borkowski, T., et al., J. Exp. Med. 184:2417 (1996)), but TGFβ1 inhibits the inflammatory stimuli-mediated activation of immature LC (Geissmann, F., J. Immunol. 162:4567-4575 (1999); Garbe A, et al., Blood, American Society of Hematology, 40th meeting, Abstract 668 (1998)). Interestingly, our results suggest a role for retinoids in the activation of immature LC in Malpighian epithelia (where most pathogens are encountered), as upon their addition, immature LC are synergistically activated in the presence of moderate amount of inflammatory mediators-1 to 10 ng/ml of TNFα or IL-1β-likely to be produced at the site of local inflammation. This may allow cell damage to be taken into account at the earliest phase of the cognate immune response by efficiently triggering an antigen-specific T-lymphocyte-mediated immune response.

In contrast, in the absence of inflammatory stimuli, natural retinoids on their own do not activate immature LC but instead increase their rate of apoptosis in a dose-dependent manner. Thus, under excess conditions, retinoids could be inefficient or even have an inhibitory effect to mount an immune response in situations where inflammatory stimuli are weak or absent. Similar effects of retinoids (apoptosis in the absence of inflammatory stimuli and enhancement of TNFα-induced activation) have been observed in an other model of DC obtained from human monocytes after culture in the presence of GM-CSF and IL-4 (Sallusto, F. and Lanzavecchia, A., J. Exp. Med. 179:1109-1119 (1994); our unpublished data). Therefore the opposite effects of retinoids on the immune response that have been described both in vitro and in vivo (Underwood, B. A. and Arthur, P., FASEB J. 10:1040-1048 (1996); Semba, R. D., Clin. Infect. Dis. 19:489-499 (1994); Rahmathullah, L., et al., N. Eng. J. Med. 323:929-935 (1990); Sommer, A., Lancet 339:864 (1992); Ross, A. C. and Stephensen, C. B., FASEBJ. 10:979-985 (1996); Semba, R. D. et al., Lancet. 345:1330-1332 (1995); Dresser, D. W., Nature 217:527-529 (1968); Malkovsky, M., et al., Nature 302:338-340 (1983); Bedford, P. A. and Knight, S. C., Clin. Exp. Immunol. 75:481-486 (1989); Katz, D. R., et al., Br. J. Exp. Path. 68:343-350 (1987); Walsh, L. J., et al., J. Invest Dermatol. 85:501-506 (1985); Meunier, L., et al., J. Invest. Dermatol. 103:775-779 (1994); Hachisuka, H. and Uno, H., Am. J. Dermatopathol. 9:316-323 (1987); Williams, N. A. and Hill, T. J., J. Invest. Dermatol. 97:933-937 (1991); Semba, R. D., et al., Public Health 111:245-247 (1997)) could be related to the presence and absence of inflammatory signals.

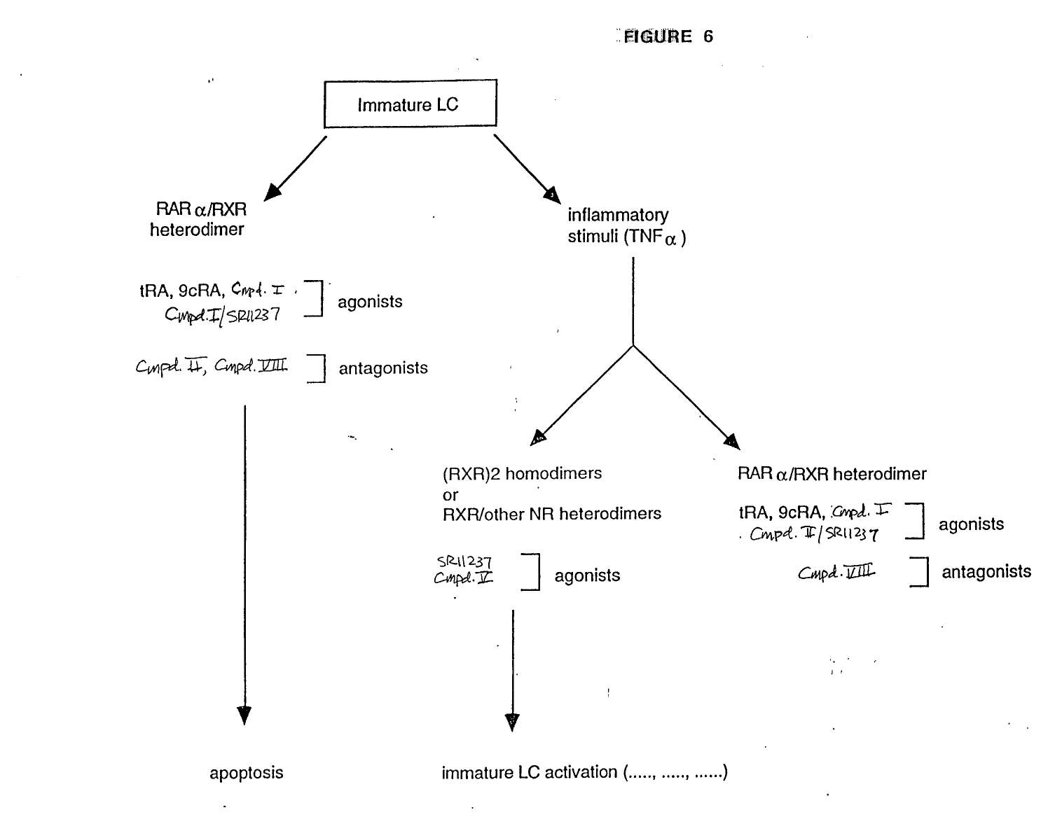

Our results demonstrate that the retinoid signal triggering caspase-dependent immature LC apoptosis in the absence of inflammatory signals is transduced by RARα/RXR heterodimers in which the transcriptional activity of RXR upon binding of an agonistic ligand is subordinated (Chen, J. Y., et al., Nature 382:819-822 (1996); Chen, J. Y., et al., EMBO J. 14:1187-1197 (1995); Vivat, V., et al., EMBO J. 16:5697-5709 (1997)) to the binding of agonist ligand to its RARα partner (FIG. 1, Table 3, FIG. 6). On the other hand, the retinoid signals that synergize with TNFα to induce LC activation (as evidenced by increased DNA-binding activity of NF-κB complexes, increase of cell surface expression of class II and costimulatory molecules, and enhancement of antigen presentation) are mediated by two distinct pathways that can be dissociated from the apoptotic one by using synthetic ligands (see FIG. 6). A first pathway is RXR-dependent and RAR-independent, as it is induced by RXR-selective agonists (SR11237, Compound V) and unaffected by RAR antagonists (Compound VIII, Compound II). The retinoid signal may be transduced by RXR homodimers acting on DR1 response elements, and/or possibly through heterodimers between RXR and other nuclear receptors, such as PPARs or several orphan receptors (Chambon, P., FASEB. J. 10:940-954 (1996); Mangelsdorf, D. J. and Evans, R. M., Cell 83:841-850 (1995); Forman, B. M., et al., Cell 83:803-312 (1995); Kliewer, S. A., et al., Cell 83:813-819 (1995)). A second pathway involves RARα/RXR heterodimers as evidenced by the induction of LC activation by the RARα selective agonist Compound I, and the synergistic effect of the RARα-selective ligand Compound II and the RXR selective agonist SR11237 (see FIGS. 3C-E). In this respect, note that Compound II, which. inhibits transactivation by RARα from DR5 response elements in transfection experiments, was already shown in another instance (Chen, J. Y., et al., Nature 382:819-822 (1996)) to synergize with the RXR agonist SR11237 via RARα/RXR heterodimers. However, this second RARα/RXR heterodimer pathway is clearly different from the heterodimer pathway responsible for apoptosis of immature LC in the absence of TNFα, as the latter is not triggered by the Compound II/SR11237 combination. Thus, the ligand-binding requirements of RARα/RXR heterodimers to induce apoptosis and activation of immature LC are different (FIG. 6), indicating that the responsive genes and coactivators proteins involved in these two processes are most probably different.

The availability of selective retinoid nuclear receptor ligands, which dissociate apoptosis and activation of immature LC, may open new avenues for the therapeutic control of immune response, e.g., to improve efficiency of vaccination against certain pathogens or tumor antigen. For example, a RXR selective agonist (SR11237) has no effect on its own on LC apoptosis, whereas it can induce LC activation, and the RARα ligand Compound II in combination with the RXR agonist SR11237 stimulates antigen presentation in the presence of inflammatory cytokines (FIG. 4), while it inhibits the RARα/RXR-mediated apoptotic effects of natural and synthetic retinoids (Table 3).

Vitamin A deficiency impairs resistance to infection and increases the risk of death, particularly to pathogens encountered at epithelial barriers, and induces a broad immune defect in response to infection, involving T-, B-, and NK-cells (Underwood, B. A. and Arthur, P., FASEB J. 10: 1040-1048 (1996); Semba, R. D., Clin. Infect. Dis. 19:489-499 (1994); Rahmathullah, L., et al., N. Eng. J. Med. 323:929-935 (1990); Sommer, A., Lancet 339:864 (1992); Ross, A. C. and Stephensen, C. B., FASEB J. 10:979-985 (1996)). In our study, the minimum effective dose of retinol for significant LC activation (1 μM) closely corresponds to the serum threshold for vitamin A deficiency (1.05 μM) (Semba, R. D., Clin. Infect. Dis. 19:489-499 (1994)). Thus, a retinoid-related defect in immature dendritic cell activation offers a plausible explanation for the immune defect and the increased mortality due to infection in vitamin A deficiency. Furthermore, increased susceptibility to infection of some individuals in Western countries could be due, in some cases, to alteration or polymorphisms in vitamin A metabolism.

Having now fully described the present invention in some detail by way of illustration and example for purposes of clarity of understanding, it will be apparent to one of ordinary skill in the art that the same can be performed by modifying or changing the invention within a wide and equivalent range of conditions, formulations and other parameters without affecting the scope of the invention or any specific embodiment thereof, and that such modifications or changes are intended to be encompassed within the scope of the appended claims.

All publications, patents and patent applications mentioned in this specification are indicative of the level of skill of those skilled in the art to which this invention pertains, and are herein incorporated by reference to the same extent as if each individual publication, patent or patent application was specifically and individually indicated to be incorporated by reference.

What is claimed is:

1. A method of modulating the immune system of an animal by affecting the physiology of an antigen-presenting cell in said animal, comprising contacting said antigen-presenting cell with an effective amount of at least one retinoid and an effective amount of at least one cytokine, under conditions whereby the physiology of said antigen-presenting cell is affected.

2. The method of claim 1, wherein the effect upon said antigen-presenting cell is activation of said cell.

3. The method of claim 1, wherein the effect upon said antigen-presenting cell is inhibition, delaying, or prevention of apoptosis in said cell.

4. The method of claim 2, wherein said retinoid is selected from the group consisting of a pan-RXR agonist and an RAR antagonist.

5. The method of claim 4, wherein said pan-RXR agonist is selected from the group consisting of SR11237, Compound V, and pharmaceutically acceptable salts, esters and prodrugs thereof.

6. The method of claim 4, wherein said RAR antagonist is selected from the group consisting of Compound II, Compound V, Compound VIII, and pharmaceutically acceptable salts, esters and prodrugs thereof.

7. The method of claim 3, wherein said retinoid is an RAR agonist.

8. The method of claim 7, wherein said RAR agonist is an RARα agonist.

9. The method of claim 8, wherein said RARα agonist is Compound I or a pharmaceutically acceptable salt, ester or prodrug thereof.

10. The method of claim 1, wherein said cytokine is selected from the group consisting of TNFα, IL1-β, and active fragments, variants, analogues and derivatives thereof.

11. A method of inducing apoptosis in a mammalian antigen-presenting cell, comprising contacting said cell with an effective amount of at least one synthetic retinoid under conditions whereby said antigen-presenting cell is induced to undergo apoptosis.

12. The method of claim 11, wherein said synthetic retinoid is selected from the group consisting of an RARα agonist, an RARβ agonist, and a pan-RXR agonist.

13. The method of claim 12, wherein said RARα agonist is Compound I or a pharmaceutically acceptable salt, ester or prodrug thereof.

14. The method of claim 12, wherein said RARβ agonist is selected from the group consisting of Compound III , Compound VII, and pharmaceutically acceptable salts, esters and prodrugs thereof.

15. The method of claim 12, wherein said pan-RXR agonist is selected from the group consisting of SR11237, Compound V, and pharmaceutically acceptable salts, esters and prodrugs thereof.

16. The method of claim 1 or claim 11, wherein said antigen-presenting cell is a dendritic cell or a Langerhans cell.

17. A composition for use in modulating the immune system of an animal comprising at least one retinoid and at least one cytokine, each being present in said composition in an amount effective to modulate the immune system of said animal.

18. The composition of claim 17, wherein said modulation is accomplished by activating an antigen-presenting cell in said animal.

19. The composition of claim 17, wherein said modulation is accomplished by inhibiting, delaying or preventing the apoptosis of an antigen-presenting cell in said animal.

20. The composition of claim 18, wherein said retinoid is selected from the group consisting of a pan-RXR agonist and an RAR antagonist.

21. The composition of claim 20, wherein said pan-RXR agonist is selected from the group consisting of SR11237, Compound V, and pharmaceutically acceptable salts, esters and prodrugs thereof.

22. The composition of claim 20, wherein said RAR antagonist is selected from the group consisting of Compound II, Compound V, Compound VIII, and pharmaceutically acceptable salts, esters and prodrugs thereof.

23. The composition of claim 19, wherein said retinoid is an RAR agonist.

24. The composition of claim 23, wherein said RAR agonist is an RARα agonist.

25. The composition of claim 24, wherein said RARα agonist is Compound I or a pharmaceutically acceptable salt, ester or prodrug thereof.

26. The composition of claim 17, wherein said cytokine is selected from the group consisting of TNFα, IL1-β, and active fragments, variants, analogues and derivatives thereof.

27. The composition of claim 17, further comprising one or more antigens.

28. The composition of claim 27, wherein said one or more antigens are selected from the group consisting of one or more bacterial antigens, one or more fungal antigens, one or more viral antigens, one or more animal antigens, one or more tumor cell antigens, one or more plant antigens, and combinations thereof.

29. The composition of claim 17 or claim 27, further comprising one or more pharmaceutically acceptable carriers or excipients.

30. A composition for use in inducing apoptosis in a mammalian antigen-presenting cell, comprising a pharmaceutically acceptable carrier and at least one synthetic retinoid selected from the group consisting of an RARα agonist, an RARβ agonist, and a pan-RXR agonist.

31. The composition of claim 30, wherein said RARα agonist is Compound I or a pharmaceutically acceptable salt, ester or prodrug thereof.

32. The composition of claim 30, wherein said RARβ agonist is selected from the group consisting of Compound III, Compound VII, and pharmaceutically acceptable salts, esters and prodrugs thereof.

33. The composition of claim 30, wherein said pan-RXR agonist is selected from the group consisting of SR11237, Compound V, and pharmaceutically acceptable salts, esters and prodrugs thereof.

34. The composition of any one of claims 17, 27 and 30, wherein said antigen-presenting cell is a dendritic cell or a Langerhans cell.

35. A method for treating or preventing a physical disorder in an animal, comprising administering to an animal suffering from, or predisposed or susceptible to, said physical disorder an effective amount of the composition of any one of claims 17, 27 and 30.

36. The method of claim 35, wherein said physical disorder is selected from the group consisting of an infectious disease, a parasitic disease, an immune system dysfunction and a cancer.

37. The method of claim 35, wherein said animal is a human.
